(19) United States
(12) Patent Application Publication
(10) Pub. No.: US 2014/0018380 A1
(43) Pub. Date: Jan. 16, 2014

(54) BICYCLIC HETEROARYL COMPOUNDS AS GPR119 RECEPTOR AGONISTS

(71) Applicants: Dengming Xiao, Beijing (CN); Yan Zhu, Beijing (CN); Yuandong Hu, Beijing (CN); Huting Wang, Beijing (CN); Yuliang Liu, Beijing (CN); Jijun Li, Beijing (CN); Deguang Sun, Beijing (CN); Zhe Wang, Beijing (CN); Yongheng Wei, Beijing (CN); Zanping Wang, Beijing (CN); Guojing Tang, Beijing (CN); Lutao Jing, Beijing (CN)

(72) Inventors: Dengming Xiao, Beijing (CN); Yan Zhu, Beijing (CN); Yuandong Hu, Beijing (CN); Huting Wang, Beijing (CN); Yuliang Liu, Beijing (CN); Jijun Li, Beijing (CN); Deguang Sun, Beijing (CN); Zhe Wang, Beijing (CN); Yongheng Wei, Beijing (CN); Zanping Wang, Beijing (CN); Guojing Tang, Beijing (CN); Lutao Jing, Beijing (CN)

(73) Assignee: CENTAURUS BIOPHARMA CO., LTD., Beijing (CN)

(21) Appl. No.: 14/003,174

(22) PCT Filed: Jan. 31, 2012
(86) PCT No.: PCT/CN2012/070800

§ 371 Date: Sep. 4, 2013

Related U.S. Application Data
(60) Provisional application No. 61/457,448, filed on Mar. 30, 2011.

(30) Foreign Application Priority Data
Jan. 31, 2011 (CN) 201110034156.8

(51) Int. Cl. C07D 401/04; C07D 471/04; C07D 413/14; C07D 401/14

(52) U.S. Cl. CPC C07D 401/04; C07D 401/14; C07D 471/04; C07D 413/14
USPC 514/275; 544/331; 514/323; 546/201; 435/375

(57) ABSTRACT

The present invention provides a new class of bicyclic heteroaryl compounds represented by Formula I, pharmaceutical compositions containing these compounds, and their use for modulating the activity of GPR119 in the treatment of metabolic disorders and complications thereof, as well as methods for the treatment of the metabolic disorders and complications thereof.

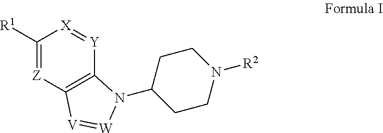

### FIELD OF THE INVENTION

The present invention relates to a new class of bicyclic heteroaryl compounds, pharmaceutical compositions containing these compounds, and their use for modulating the activity of GPR119 in the treatment of metabolic disorders and complications thereof.

### BACKGROUND ART

Diabetes mellitus is a serious illness afflicting millions of people across the world. The most common forms of diabetes mellitus are Type I (also referred to insulin-dependent diabetes mellitus) and Type II diabetes (also referred to non-insulin-dependent diabetes mellitus). Type II diabetes, accounting for roughly 90% of all diabetic cases, is a serious progressive disease that results in microvascular complications (including retinopathy, neuropathy and nephropathy) as well as macrovascular complications (including accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin, thiazolidinediones, or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion only in the presence of high glucose, but must be injected due to a lack of oral bioavailablity. Sitagliptin, a dipeptidyl peptidase IV inhibitor, is a new drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

In Type II diabetes, muscle, fat and liver cells fail to respond normally to insulin. This condition (insulin resistance) may be due to reduced numbers of cellular insulin receptors, disruption of cellular signaling pathways, or both. At first, the beta cells compensate for insulin resistance by increasing insulin output. Eventually, however, the beta cells become unable to produce sufficient insulin to maintain normal glucose levels (euglycemia), indicating progression to Type II diabetes.

In Type II diabetes, fasting hyperglycemia occurs due to insulin resistance combined with beta cell dysfunction. There are two aspects of beta cell defect dysfunction: 1) increased basal insulin release (occurring at low, non-stimulatory glucose concentrations). This is observed in obese, insulin-resistant pre-diabetic stages as well as in Type II diabetes, and 2) in response to a hyperglycemic challenge, a failure to increase insulin release above the already elevated basal level. This does not occur in pre-diabetic stages and may signal the transition from normo-glycemic insulin-resistant states to frank Type II diabetes. Current therapies to treat the latter aspect include inhibitors of the beta-cell ATP-sensitive potassium channel to trigger the release of endogenous insulin stores, and administration of exogenous insulin. Neither achieves accurate normalization of blood glucose levels and both expose to the risk of eliciting hypoglycemia.

Thus, there is a great interest in the discovery of agents that function in a glucose-dependent manner. Physiological signaling pathways which function in this way are well known, including gut peptides GLP-1 and GIP. These hormones signal via cognate G-protein coupled receptors to stimulate production of cAMP in pancreatic beta-cells. Increased cAMP apparently does not result in stimulation of insulin release during the fasting or pre-prandial state. However, a number of biochemical targets of cAMP, including the ATP-sensitive potassium channel, voltage-sensitive potassium channels and the exocytotic machinery, are modulated such that insulin secretion due to postprandial glucose stimulation is significantly enhanced. Therefore, agonist modulators of novel, similarly functioning, beta-cell GPCRs, including GPR119, would also stimulate the release of endogenous insulin and promote normalization of glucose levels in Type II diabetes patients. It has also been shown that increased cAMP, for example as a result of GLP-1 stimulation, promotes beta-cell proliferation, inhibits beta-cell death and thus improves islet mass. This positive effect on beta-cell mass should be beneficial in Type II diabetes where insufficient insulin is produced.

Many people with Type II diabetes have sedentery lifestyles and are obese; they weigh about 20% more than the recommended weight for their height and build. Obesity is characterized by hyperinsulinemia, insulin resistance, hypertension and atherosclerosis.

Obesity and diabetes are among the most common health problems in industrialized societies. In industrialized countries, more that 20% people are overweight. Obesity, which is the result of an imbalance between caloric intake and energy expenditure, is highly correlated with insulin resistance and diabetes in experimental animals and human. Obesity is one of the most important risk factors for Type II diabetes.

It is well known that metabolic diseases have negative effects on other physiological systems and there is often co-occurrence of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy. Thus, treatment of the diabetic condition should be of benefit to such interconnected disease states.

### SUMMARY OF THE INVENTION

The present invention relates to compounds which are activators of the GPR119 receptors, or GPR119 receptor agonists, and are useful in the treatment of metabolic diseases and disorders, in particular for Type II diabetes.

These compounds may be represented by Formula I, as shown below:

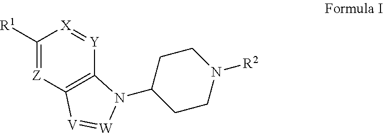

wherein:

R¹ is aryl, unsubstituted or substituted with one or more substituents selected from the group consisting of halogen, alkyl (preferably lower alkyl), alkoxy (preferably lower alkoxy), OCF₃, alkoxycarbonyl, cyano, NHC(O)-alkyl, SO₂-alkyl, SO₂-cyclo alkyl, SO₂NH₂, SO₂NH-alkyl, —N(alkyl)-SO₂-alkyl, C(O)-alkyl, NO₂, NHS(O)₂-alkyl, SO₂N-(alkyl)₂, CONH-alkyl, CON-(alkyl)₂, S(O)-alkyl, S(O)-cycloalkyl, C(O)NH₂, triazole, tetrazole, acetyl-piperazine, unsubstituted monocyclic heteroaryl and monocyclic heteroaryl substituted with alkyl;

1,1-dioxo-2,3-dihydro-1H-1-benzo[b]thiophenyl;

monocyclic heteroaryl, unsubstituted or substituted with one or more substituents selected from the group consisting of halogen, SO₂-alkyl, SO₂-cycloalkyl, lower alkyl, triazole, tetrazole, monocyclic heteroaryl with one or two heteroatoms selected from the group consisting of N, O and S; oxo, alkoxy, cyano and hydroxyl; indole, unsubstituted or substituted with one or more substituents selected from the group consisting of lower alkyl, oxo, triazole, tetrazole, SO₂-alkyl and SO₂-cycloalkyl;

benzo[1,3]dioxole, unsubstituted or substituted with one or more substituents selected from the group consisting of alkyl, triazole, tetrazole, oxo, SO₂-alkyl, and SO₂-cycloalkyl;

quinoline, unsubstituted or substituted with one or more substituents selected from the group consisting of lower alkyl, oxo, triazole, tetrazole, SO₂-alkyl and SO₂-cycloalkyl;

pyrrolo[2,3-b]pyridine, unsubstituted or substituted with one or more substituents selected from the group consisting of lower alkyl, oxo, triazole, tetrazole, SO₂-alkyl and SO₂-cycloalkyl;

benzothiophene, unsubstituted or substituted with one or more substituents selected from the group consisting of lower alkyl, oxo, SO₂-alkyl and SO₂-cycloalkyl; or

dioxobenzothiophene, unsubstituted or substituted with one or more substituents selected from the group consisting of lower alkyl, oxo, triazole, tetrazole, SO₂-alkyl and SO₂-cycloalkyl;

R² is benzyl, unsubstituted or substituted with one or more substituents selected from the group consisting of cyano, alkoxy, halogen, hydroxy, OCF₃ and CF₃;

C(O)—O-alkyl;

C(O)—O—(CH₂)-cycloalkyl;

C(O)—O—(CH₂)ₙ-phenyl, said phenyl being unsubstituted or substituted with halogen, CF₃, cyano or NO₂;

heteroaryl, unsubstituted or substituted with the substituents selected from the group consisting of halogen, lower alkyl, cycloalkyl or alkoxy;

(CH₂)-heteroaryl, said heteroaryl being unsubstituted or substituted with the substituents selected from the group consisting of halogen, lower alkyl, cycloalkyl or alkoxy;

C(O)-lower alkyl;

C(O)(CH₂)-cycloalkyl;

C(O)(CH₂)ₙ-phenyl, said phenyl being unsubstituted or substituted with halogen or alkoxy;

C(O)-heteroaryl, said heteroaryl being unsubstituted or substituted with halogen, lower alkyl or alkoxy;

C(O)-aryl, said aryl being unsubstituted or substituted with halogen, lower alkyl or alkoxy;

CH₂-difluorobenzodioxole; or

SO₂-lower alkyl; and

n is 0, 1 or 2;

in the moiety of

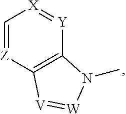

X, Y, Z, V and W are independently selected from N, or CR³; and the moiety is optionally substituted with one or more substituents selected from halogen, cyano, optionally substituted alkyl (preferably C₁₋₆ alkyl), cycloalkyl (preferably C₃₋₅ cycloalkyl) and alkoxy (preferably C₁₋₆ alkoxy). Preferably, the substituents are bonded to a carbon ring atom.

R³ is hydrogen, halogen, alkyl (preferably lower alkyl), hydroxy or alkoxy (preferably lower alkyl);

or a pharmaceutically acceptable salt, solvate, poly-morph, tautomer or prodrug thereof.

In another aspect, the present invention provides some preferable compounds of Formula I, wherein in R¹, the aryl is monocyclic aryl, and phenyl is preferred; and in the substitutents and in R³, each alkyl is C₁₋₆ alkyl, and —CH₃ or —CH₂CH₃ is preferred; each cycloalkyl is C₃₋₅ cycloalkyl, and each alkoxy is C₁₋₆ alkoxy; or a pharmaceutically acceptable salt, solvate, poly-morph, tautomer or prodrug thereof.

In another aspect, the present invention provides some preferable compounds of Formula I, wherein in R², the heteroaryl is monocyclic heteroaryl with at least one heteroatoms of N, S and O; and in the substitutents, each alkyl is C₁₋₆ alkyl, each cycloalkyl is C₃₋₅ alkyl, and each alkoxy is C₁₋₆ alkoxy; or a pharmaceutically acceptable salt, solvate, poly-morph, tautomer or prodrug thereof.

In another aspect, the present invention is a subclass of compounds of formula (I), represented by the following formula (II):

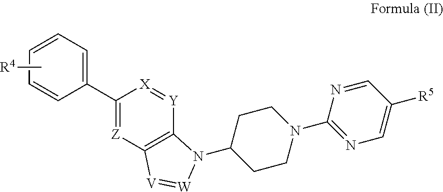

wherein, R⁴ is at least one group selected from the group consisting of —SO₂C₁₋₄ alkyl, cycloalkyl, —NHS(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen; and each alkyl above is preferably methyl.

R⁵ is C₁₋₄alkyl, C₁₋₄alkoxy, halogen, C₃₋₆cycloalkyl or heterocyclic;

or a pharmaceutically acceptable salt, solvate, poly-morph, tautomer or prodrug thereof.

In another aspect, the present invention provides some preferable compounds of Formula II, wherein R⁴ is one group at the ortho-position, meta-position or para-position to the other substituent of phenyl; or a pharmaceutically acceptable salt, solvate, poly-morph, tautomer or prodrug thereof.

In another aspect, the present invention is a subclass of compounds of formula (I), represented by the following formula (III):

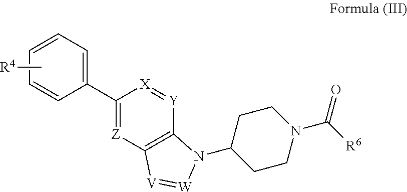

wherein, R⁴ is at least one group selected from the group consisting of —SO₂alkyl, —SO₂ cycloalkyl, —NH S(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen, preferably, the “alkyl” alone or in combination used in R⁴ is preferably C₁₋₆ alkyl, more preferably C₁₋₄ alkyl (methyl is preferred); and R⁶ is C₁₋₆ alkyl, monocyclic aryl, monoheteroaryl, C₁₋₆ alkoxy, C₃₋₆ cycloalkyl (for example, C₃₋₅ cycloalkyl) or heterocyclic (for example, C₁₋₆ or C₃₋₆ heterocyclic with one or more heteroatoms selected from N, O and S); or a pharmaceutically acceptable salt, solvate, poly-morph, tautomer or prodrug thereof.

In another aspect, the invention provides some preferable compounds of Formula III, wherein R⁴ is one group at the ortho-position, meta-position or para-position to the other substituent of phenyl; or a pharmaceutically acceptable salt, solvate, poly-morph, tautomer or prodrug thereof.

In another aspect, the present invention is a subclass of compounds of formula (I), represented by the following formula (IV):

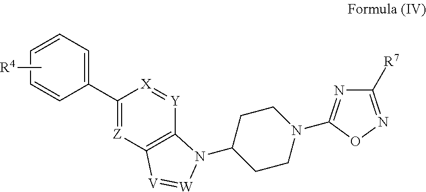

wherein, R⁴ is at least one group selected from the group consisting of —SO₂ alkyl, —SO₂cycloalkyl, —NHS(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen, preferably, the “alkyl” alone or in combination used in R⁴ is preferably C₁₋₆ alkyl, more preferably C₁₋₄ alkyl, for example methyl etc.; and R⁷ is C₁₋₆ alkyl (C₁₋₄alkyl is preferred), C₁₋₆ alkoxy (C₁₋₄alkoxy is preferred), halogen, monocyclic aryl, monoheteroaryl, C₃₋₆ cycloalkyl (for example, C₃₋₅ cycloalkyl) or heterocyclic (for example, C₁₋₆ or C₃₋₆ heterocyclic with one or more heteroatoms selected from N, O and S); or a pharmaceutically acceptable salt, solvate, poly-morph, tautomer or prodrug thereof.

In another aspect, the invention provides some preferable compounds of Formula IV, wherein R⁴ is one group at the ortho-position, meta-position or para-position to the other substituent of phenyl; or a pharmaceutically acceptable salt, solvate, poly-morph, tautomer or prodrug thereof.

In another aspect, the invention provides some preferable compounds of the present invention, wherein

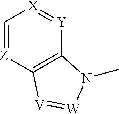

may be selected from

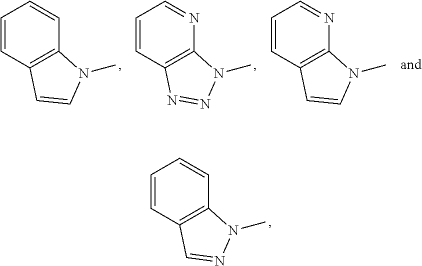

etc.

In another aspect, the present invention provides the compound represented by any of the following formula or a pharmaceutically acceptable salt, solvate, poly-morph, tautomer or prodrug thereof:

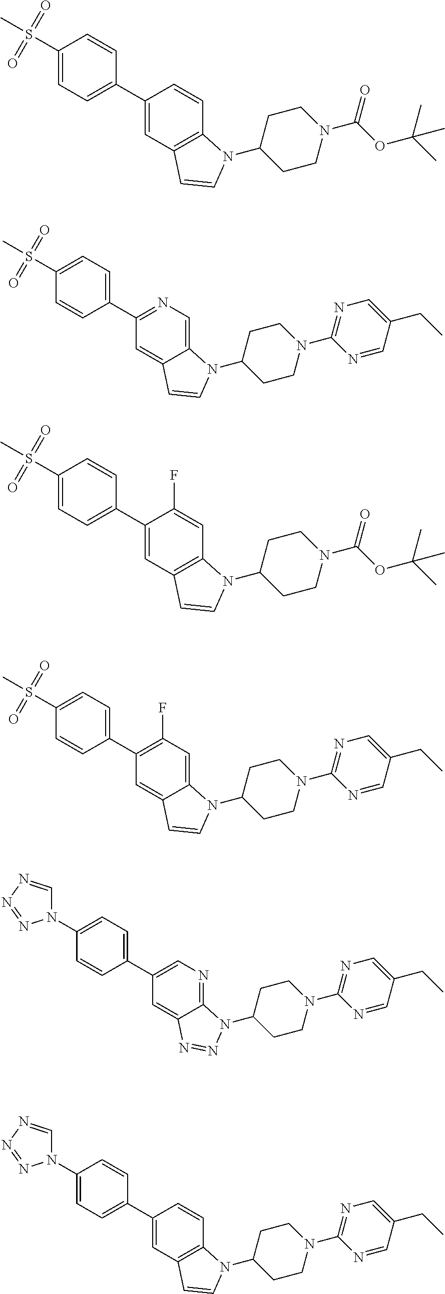

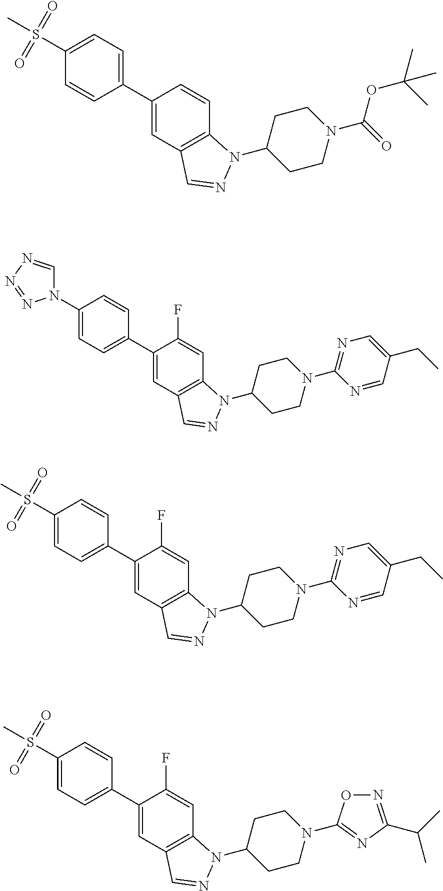

In another aspect, the present invention is directed to a compound above mentioned or a pharmaceutically acceptable salt, solvate, poly-morph, tautomer or prodrug thereof, for use as a GPR119 receptor agonist.

In another aspect, the present invention is directed to a compound above mentioned or a pharmaceutically acceptable salt, solvate, poly-morph, tautomer or prodrug thereof, for use as a medicament for the treatment of a metabolic-related disorder. Preferably, said metabolic-related disorder is selected from the group consisting of Type I diabetes, Type II diabetes, inadquate glucose tolerance, insulin resistance, hyperglycemia, hyperlipidemia, hypertriglyceridemia, hypercholesterolemia, dyslipidemia, obesity and syndrome X.

In another aspect, the present invention is directed to a pharmaceutical composition comprising an effective amount of a compound of this invention or a pharmaceutically acceptable salt, solvate, poly-morph, tautomer or prodrug thereof. In some embodiments, the pharmaceutical composition further comprises a pharmaceutically acceptable carrier. Such a composition may contain, as a pharmaceutically acceptable carrier, at least one of adjuvants, excipients, and preservatives, agents for delaying absorption, fillers, binders, adsorbents, buffers, disintegrating agents, solubilizing agents, and other inert ingredients. Methods of formulating the composition are well-known in the art.

In another aspect, the present invention is directed to a method for stimulating the release of endogenous insulin from an isolet beta-cell comprising the contact of a compound of this invention or a pharmaceutically acceptable salt, solvate, poly-morph, tautomer or prodrug thereof with the cell. In an embodiment, the cell is in vitro. In another embodiment, the cell is in vivo.

In another aspect, the present invention is directed to a method for the treatment of a metabolic-related disorder in an individual comprising administering to said individual in need of such treatment a therapeutically effective amount of a compound of this invention or a pharmaceutically acceptable salt, solvate, poly-morph, tautomer or prodrug thereof. Preferably, the individual is a mammal; and more preferably, the individual is a human. In some embodiments, said metabolic-related disorder is selected from the group consisting of Type I diabetes, Type II diabetes, inadquate glucose tolerance, insulin resistance, hyperglycemia, hyperlipidemia, hypertriglyceridemia, hypercholesterolemia, dyslipidemia, obesity and syndrome X. The appropriate dosage for a particular patient can be determined, according to known methods, by those skilled in the art.

In another aspect, the present invention is directed to use of a compound of this invention or a pharmaceutically acceptable salt, solvate, poly-morph, tautomer or prodrug thereof in the preparation of a medicament used as a GPR119 receptor agonist.

In another aspect, the present invention is directed to use of a compound of this invention or a pharmaceutically acceptable salt, solvate, poly-morph, tautomer or prodrug thereof in the preparation of a medicament for the treatment of a metabolic-related disorder. In some embodiments, said metabolic-related disorder is selected from the group consisting of Type I diabetes, Type II diabetes, inadquate glucose tolerance, insulin resistance, hyperglycemia, hyperlipidemia, hypertriglyceridemia, hypercholesterolemia, dyslipidemia, obesity and syndrome X.

In another aspect, the present invention is directed to a pharmaceutical composition comprising a compound of this invention or a pharmaceutically acceptable salt, solvate, polymorph, tautomer or prodrug thereof. In some embodiments, the pharmaceutical composition is in a form suitable for oral administration, parenteral administration, topical administration and rectal administration, etc. In further or additional embodiments, the pharmaceutical composition is in the form of a tablet, capsule, pill, powder, sustained release formulation, solution and suspension, for parenteral injection as a sterile solution, suspension or emulsion, for topical administration as an ointment or cream or for rectal administration as a suppository. In further or additional embodiments, the pharmaceutical composition is in unit dosage forms suitable for single administration of precise dosages. In further or additional embodiments the amount of compound of formula I is in the range of about 0.001 to about 1000 mg/kg body weight/day. In further or additional embodiments the amount of compound of formula I is in the range of about 0.5 to about 50 mg/kg body weight/day. In further or additional embodiments the amount of compound of formula I is about 0.001 to about 7 g/day. In further or additional embodiments the amount of compound of formula I is about 0.002 to about 6 g/day. In further or additional embodiments the amount of compound of formula I is about 0.005 to about 5 g/day. In further or additional embodiments the amount of compound of formula I is about 0.01 to about 5 g/day. In further or additional embodiments the amount of compound of formula I is about 0.02 to about 5 g/day. In further or additional embodiments the amount of compound of formula I is about 0.05 to about 2.5 g/day. In further or additional embodiments the amount of compound of formula I is about 0.1 to about 1 g/day. In further or additional embodiments, dosage levels below the lower limit of the aforesaid range may be more than adequate. In further or additional embodiments, dosage levels above the upper limit of the aforesaid range may be required. In further or additional embodiments the compound of formula I is administered in a single dose, once daily. In further or additional embodiments the compound of formula I is administered in multiple doses, more than once per day. In further or additional embodiments the compound of formula I is administered twice daily. In further or additional embodiments the compound of formula I is administered three times per day. In further or additional embodiments the compound of formula I is administered four times per day. In further or additional embodiments the compound of formula I is administered more than four times per day. In some embodiments, the pharmaceutical composition is for administration to a mammal. In further or additional embodiments, the mammal is human. In further or additional embodiments, the pharmaceutical composition further comprises a pharmaceutical carrier, excipient and/or adjuvant. In further or additional embodiments, the pharmaceutical composition further comprises at least one therapeutic agent. In further or additional embodiments, the therapeutic agent is a drug for treating a diabetes.

In some embodiments, the composition comprising a compound of formula I is administered orally, intraduodenally, parenterally (including intravenous, subcutaneous, intramuscular, intravascular or by infusion), topically or rectally. In some embodiments, the pharmaceutical composition is in a form suitable for oral administration. In further or additional embodiments, the pharmaceutical composition is in the form of a tablet, capsule, pill, powder, sustained release formulations, solution and suspension, for parenteral injection as a sterile solution, suspension or emulsion, for topical administration as an ointment or cream or for rectal administration as a suppository. In further or additional embodiments, the pharmaceutical composition is in unit dosage forms suitable for single administration of precise dosages. In further or additional embodiments, the pharmaceutical composition further comprises a pharmaceutical carrier, excipient and/or adjuvant. In some embodiments, the individual is a mammal. In further or additional embodiments, the individual is a human. In some embodiments, the composition comprising a compound of formula I is administered in combination with an additional therapy.

In another aspect, the present invention is directed to a process for preparing a compound of formula I or a pharmaceutically acceptable salt, solvate, polymorph, tautomer or prodrug thereof.

### DETAILED DESCRIPTION OF THE INVENTION

The novel features of the invention are set forth with particularity in the appended claims. A better understanding of the features and advantages of the present invention will be obtained by reference to the following detailed description that sets forth illustrative embodiments, in which the principles of the invention are utilized.

While preferred embodiments of the present invention have been shown and described herein such embodiments are provided by way of example only. It should be understood that various alternatives to the embodiments of the invention described herein may be employed in practicing the invention. Those ordinary skilled in the art will appreciate that numerous variations, changes, and substitutions are possible without departing from the invention. It is intended that the following claims define the scope of aspects of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Certain Chemical Terminology

Unless defined otherwise, all technical and scientific terms used herein have the same meaning as is commonly understood by one of skill in the art to which the claimed subject matter belongs. All patents, patent applications, published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting. Likewise, use of the term “comprising” as well as other forms, such as “comprise”, “comprises”, and “comprised” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed in connection with, and the laboratory procedures and techniques of, analytical chemistry, synthetic organic chemistry, and medicinal and pharmaceutical chemistry described herein are those known in the art. Standard techniques can be used for chemical syntheses, chemical analyses, pharmaceutical preparation, formulation, and delivery, and treatment of patients. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, CH₂O is equivalent to OCH₂.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

The compounds presented herein may possess one or more stereocenters and each center may exist in the R or S configuration, or combinations thereof. Likewise, the compounds presented herein may possess one or more double bonds and each may exist in the E (trans) or Z (cis) configuration, or combinations thereof. Presentation of one particular stereoisomer, regioisomer, diastereomer, enantiomer or epimer should be understood to include all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. Techniques for inverting or leaving unchanged a particular stereocenter, and those for resolving mixtures of stereoisomers are well known in the art and it is well within the ability of one of skill in the art to choose an appropriate method for a particular situation. See, for example, Fumiss et al. (eds.), VOGEL'S ENCYCLOPEDIA OF PRACTICAL ORGANIC CHEMISTRY 5.sup.TH ED., Longman Scientific and Technical Ltd., Essex, 1991, 809-816; and Heller, Acc. Chem. Res. 1990, 23, 128.

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

The term “catalytic group” refers to a chemical functional group that assists catalysis by acting to lower the activation barrier to reaction.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. Further, an optionally substituted group may be un-substituted (e.g., CH₂CH₃), fully substituted (e.g., CF₂CF₃), mono-substituted (e.g., CH₂CH₂F) or substituted at a level anywhere in-between fully substituted and mono-substituted (e.g., CH₂CHF₂, CF₂CH₃, CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons (except in those instances where macromolecular substituents are clearly intended, e.g., polypeptides, polysaccharides, polyethylene glycols, DNA, RNA and the like).

As used herein, C₁-Cn, includes C₁-C₂, C₁-C₃ . . . C₁-Cn. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, isobutyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “hydrocarbon” as used herein, alone or in combination, refers to a compound or chemical group containing only carbon and hydrogen atoms.

The terms “heteroatom” or “hetero” as used herein, alone or in combination, refer to an atom other than carbon and hydrogen. Heteroatoms are independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon monoradical having from one to about ten carbon atoms, preferably one to eight, or one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁_{_{—}}₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. The substituent(s) in the substituted alkyl is selected from the group consisting of halogen, alkyl, alkoxy, OCF₃, alkoxycarbonyl, cyano, NHC(O)-alkyl, SO₂-alkyl, SO₂-cycloalkyl, SO₂NH₂, SO₂NH-alkyl, —N(allyl)-SO₂-alkyl, C(O)-alkyl, NO₂, NHS(O)₂-alkyl, SO₂N-(alkyl)₂, CONH-alkyl, CON-(alkyl)₂, S(O)-alkyl, S(O)-cycloalkyl, C(O)NH₂, triazole, tetrazole, acetyl-piperazine, unsubstituted monocyclic heteroaryl and monocyclic heteroaryl substituted with alkyl.

The term “lower alkyl” as used herein, alone or in combination, refers to an optionally substituted straight-chain, or optionally substituted branched-chain saturated hydrocarbon monoradical having from one to about eight carbon atoms, preferably one to about six carbon atoms, more preferably one to four carbon atoms.

The term “alkyl” as used herein in combination refers to an alkyl bonding with other groups, such as the alkyl in the groups of —SO₂ alkyl, —SO₂cycloalkyl, —NHS(O)₂-alkyl, —SO₂N-(alkyl)₂, —SO₂NH₂, —SO₂NH-alkyl, —N(alkyl)-SO₂-alkyl, alkoxy, thioalkyl, hydroxyalkyl, haloalkyl, cyanoalkyl, monoalkylamino, dialkylamino, etc.

The term “alkylene” as used herein, alone or in combination, refers to a diradical derived from the above-defined monoradical, alkyl. Examples include, but are not limited to methylene (—CH₂), ethylene (—CH₂CH₂), propylene (—CH₂CH₂CH₂), isopropylene (—CH(CH₃)CH₂) and the like.

The term “alkenyl” as used herein, alone or in combination, refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (CH—CH₂), 1-propenyl (CH₂CH═CH₂), isopropenyl [C(CH₃)═CH₂], butenyl, 1,3-butadienyl and the like. Whenever it appears herein, a numerical range such as “C₂-C₆ alkenyl” or “C₂₋₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “alkenylene” as used herein, alone or in combination, refers to a diradical derived from the above-defined monoradical alkenyl. Examples include, but are not limited to ethenylene (CH—CH), the propenylene isomers (e.g., CH₂CH═CH and C(CH₃)═CH) and the like.

The term “alkynyl” as used herein, alone or in combination, refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂_{_{—}}₆ alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “alkynylene” as used herein, alone or in combination, refers to a diradical derived from the above-defined monoradical, alkynyl. Examples include, but are not limited to ethynylene (—CC—), propargylene (—CH₂CC—) and the like.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “heteroalkyl”, “heteroalkenyl” and “heteroalkynyl” as used herein, alone or in combination, refer to optionally substituted alkyl, alkenyl and alkynyl structures respectively, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof.

The terms “haloalkyl”, “haloalkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The term “perhalo” as used herein, alone or in combination, refers to groups in which all of the hydrogen atoms are replaced by fluorines, chlorines, bromines, iodines, or combinations thereof. Thus, as a non-limiting example, the term “perhaloalkyl” refers to an alkyl group, as defined herein, in which all of the H atoms have been replaced by fluorines, chlorines, bromines or iodines, or combinations thereof A non-limiting example of a perhaloalkyl group is bromo, chloro, fluoromethyl. A non-limiting example of a perhaloalkenyl group is trichloroethenyl. A non-limiting example of a perhaloalkynyl group is tribromopropynyl.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “aromatic” as used herein, refers to a planar, cyclic or polycyclic, ring moiety having a delocalized at-electron system containing 4n+2 n electrons, where n is an integer. Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an optionally substituted aromatic hydrocarbon radical of six to about twenty, or six to about ten ring carbon atoms, and includes fused and non-fused aryl rings. A fused aryl ring radical contains from two to four fused rings where the ring of attachment is an aryl ring, and the other individual rings may be alicyclic, heterocyclic, aromatic, hetero aromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “arylene” as used herein, alone or in combination, refers to a diradical derived from the above-defined monoradical, aryl. Examples include, but are not limited to 1,2-phenylene, 1,3-phenylene, 1,4-phenylene, 1,2-naphthylene and the like.

The term “heteroaryl” as used herein, alone or in combination, refers to optionally substituted aromatic mono-radicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent 0 or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. Anon-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide and the like.

The term “heteroarylene” as used herein, alone or in combination, refers to a diradical derived from the above-defined monoradical heteroaryl. Examples include, but are not limited to pyridinylene and pyrimidinylene.

The term “heterocyclyl” as used herein, alone or in combination, refers collectively to heteroalicyclyl. Herein, whenever the number of carbon atoms in a heterocycle is indicated (e.g., C₃-C₆ heterocycle), at least one non-carbon atom (the heteroatom) must be present in the ring. Designations such as “C₃-C₆ heterocycle” refer only to the number of carbon atoms in the ring and do not refer to the total number of atoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). For heterocycles having two or more heteroatoms, those two or more heteroatoms can be the same or different from one another. Heterocycles can be optionally substituted. Heterocyclyl herein includes preferably about five to about twenty, or about five to about ten, or about five to about eight, or five to six ring atoms. Bonding (i.e. attachment to a parent molecule or further substitution) to a heterocycle can be via a heteroatom or a carbon atom.

A non-limiting example of “heterocyclyl” includes azinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo [3.1.0]hexyl, 3-azabicyclo [4.1.0]heptyl, 3H-indolyl and quinolizinyl and the like. The terms also include all ring forms of the carbohydrates, including but not limited to the monosaccharides, the disaccharides and the oligosaccharides.

The term “carbocyclyl”, “carbocycle”, “cyclyl” or “cycle” as used herein, alone or in combination, refers to alicyclyl; i.e. all carbon, covalently closed ring structures, which may be saturated (i.e., cycloalkyl), partially unsaturated (i.e., cycloalkenyl). The term includes preferably about five to about twenty, or about five to about ten, or about five to about eight, or five to six ring atoms. Carbocyclic rings can be formed by three, four, five, six, seven, eight, nine, or more than nine carbon atoms. Carbocycles can be optionally substituted. The term distinguishes carbocyclic from heterocyclic rings in which the ring backbone contains at least one atom which is different from carbon.

The term “cycloalkyl” as used herein, alone or in combination, refers to an optionally substituted, saturated, hydrocarbon monoradical ring, containing from three to about fifteen ring carbon atoms or from three to about ten ring carbon atoms or from three to six carbon atoms, though may include additional, non-ring carbon atoms as substituents (e.g. methylcyclopropyl).

The terms “halogen”, “halo” or “halide” as used herein, alone or in combination refer to fluoro, chloro, bromo and iodo.

The term “alkoxy” as used herein, alone or in combination, refers to an alkyl ether radical (O-alkyl), including the groups O-aliphatic and O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tertbutoxy and the like.

The term “substituent(s)” used herein includes one or more groups substituting an optionally substituted group as defined herein. The substituent(s) is selected from the group consisting of halogen, alkyl, alkoxy, OCF₃, alkoxycarbonyl, cyano, NHC(O)-alkyl, SO₂-alkyl, SO₂-cycloalkyl, SO₂NH₂, SO₂NH-alkyl, —N(alkyl)-SO₂-alkyl, C(O)-alkyl, NO₂, NHS(O)₂-alkyl, SO₂N-(alkyl)₂, CONH-alkyl, CON-(alkyl)₂, S(O)-alkyl, S(O)-cycloalkyl, C(O)NH₂, triazole, tetrazole, acetyl-piperazine, unsubstituted monocyclic heteroaryl and monocyclic heteroaryl substituted with alkyl.

Certain Pharmaceutical Terminology

The term “subject”, “patient” or “individual” as used herein in reference to individuals suffering from a disorder, a disorder, a condition, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined using techniques, such as a dose escalation study.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In preferred embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of a compound into cells or tissues.

The term “agonist,” as used herein, refers to a molecule such as a compound, a drug, an enzyme activator or a hormone modulator which enhances the activity of another molecule or the activity of a receptor site.

The term “antagonist,” as used herein, refers to a molecule such as a compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methoxybenzo ate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate undeconate and xylenesulfonate. Other acids, such as oxalic, while not in themselves pharmaceutically acceptable, may be employed in the preparation of salts useful as intermediates in obtaining the compounds of the invention and their pharmaceutically acceptable acid addition salts (See examples at Berge et al., J. Pham. Sci. 1977, 66, 1-19.). Further, those compounds described herein which may comprise a free acid group may react with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, IV′ (C₁_{_{—}}₄ alkyl)₄, and the like. Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they may contain. Water or oil-soluble or dispersible products may be obtained by such quaternization. See, for example, Berge et al., supra.

The term “solvate” as used herein refers to a combination of a compound of this invention with a solvent molecule formed by solvation. In some situations, the solvate refers to a hydrate, i.e., the solvent molecule is a water molecule, the combination of a compound of this invention and water forms a hydrate.

The term “polymorph” or “polymorphism” as used herein refers to a compound of this invention present in different crystal lattice forms.

The term “ester” as used herein refers to a derivative of a compound of this invention derived from an oxoacid group and a hydroxyl group, either one of which can be present at the compound of this invention.

The term “tautomer” as used herein refers to an isomer readily interconverted from a compound of this invention by e.g., migration of a hydrogen atom or proton.

The term “pharmaceutically acceptable derivative or prodrug” as used herein, refers to any pharmaceutically acceptable salt, ester, salt of an ester or other derivative of a compound of this invention, which, upon administration to a recipient, is capable of providing, either directly or indirectly, a compound of this invention or a pharmaceutically active metabolite or residue thereof. Particularly favored derivatives or prodrugs are those that increase the bioavailability of the compounds of this invention when such compounds are administered to a patient (e.g., by allowing orally administered compound to be more readily absorbed into blood) or which enhance delivery of the parent compound to a biological compartment (e.g., the brain or lymphatic system).

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Various forms of prodrugs are well known in the art. See for example Design of Prodrugs, Bundgaard, A. Ed., Elseview, 1985 and Method in Enzymology, Widder, K. et al., Ed.; Academic, 1985, vol. 42, p. 309-396; Bundgaard, F L “Design and Application of Prodrugs” in A Textbook of Drug Design and Development, Krosgaard-Larsen and H. Bundgaard, Ed., 1991, Chapter 5, p. 113-191; and Bundgaard, F L, Advanced Drug Delivery Review, 1992, 8, 1-38, each of which is incorporated herein by reference. The prodrugs described herein include, but are not limited to, the following groups and combinations of these groups; amine derived prodrugs: Hydroxy prodrugs include, but are not limited to acyloxyalkyl esters, alkoxycarbonyloxyalkyl esters, alkyl esters, aryl esters and disulfide containing esters.

The terms “enhance” or “enhancing,” as used herein, means to increase or prolong either in potency or duration of a desired effect. Thus, in regard to enhancing the effect of therapeutic agents, the term “enhancing” refers to the ability to increase or prolong, either in potency or duration, the effect of other therapeutic agents on a system.

An “enhancing-effective amount,” as used herein, refers to an amount adequate to enhance the effect of another therapeutic agent in a desired system.

The terms “pharmaceutical combination”, “administering an additional therapy”, “administering an additional therapeutic agent” and the like, as used herein, refer to a pharmaceutical therapy resulting from mixing or combining more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

The terms “co-administration”, “administered in combination with” and their grammatical equivalents or the like, as used herein, are meant to encompass administration of the selected therapeutic agents to a single patient, and are intended to include treatment regimens in which the agents are administered by the same or different route of administration or at the same or different times. In some embodiments the compounds described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds of the invention and the other agent (s) are administered in a single composition.

The term “metabolite,” as used herein, refers to a derivative of a compound which is formed when the compound is metabolized.

The term “active metabolite,” as used herein, refers to a biologically active derivative of a compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

Experimental

General Methods:

All operations involving moisture and/or oxygen sensitive materials were conducted under an atmosphere of dry nitrogen in pre-dried glassware. Unless noted otherwise, materials were obtained from commercially available sources and used without further purification.

Column chromatography was performed on Qingdao Haiyang Chemical CO., LTD. silica gel (200-300 mesh). Thin layer chromatography was performed using precoated plates purchased from E. Merck (silica gel 60 PF₂₅₄, 0.25 mm).

Nuclear magnetic resonance (NMR) spectra were recorded on Varian VNMRS-400 resonance spectrometer. ¹H NMR chemical shifts are giving in parts per million (6) downfield from tetramethylsilane (TMS). ¹H NMR information is tabulated in the following format: number of protons, multiplicity (s, singlet; d, doublet; t, triplet; q, quarter; m, multiplet), coupling constant(s) (J) in Hertz.

LC/MS was taken on Mass Spectrometer on FINNIGAN Thermo LCQ Advantage MAX, Agilent LC 1200 series (Column: Waters Symmetry C18, Ø4.6×50 mm, 5 μm, 35° C.) operating in ESI(+) ionization mode.

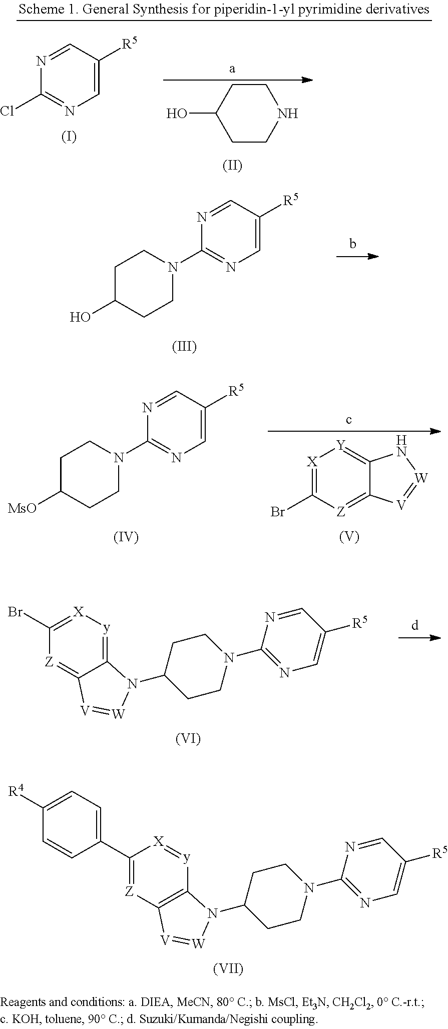

The piperidin-1-yl pyrimidine-based ligands of VII can be prepared following the general Scheme 1. Substituted piperidinpyrimidine III can be obtained from pyrimidine I and pyperidine II with the presence of DIEA. Compound III can be reacted with MsCl to afford intermediate IV. The reaction between compound IV and bromoheterocyclic compound V gives the most important intermediate VI, which can be used for making a variety of analogues. At last, the desired ligand VII was synthesized from VI through a cross-coupling reaction.

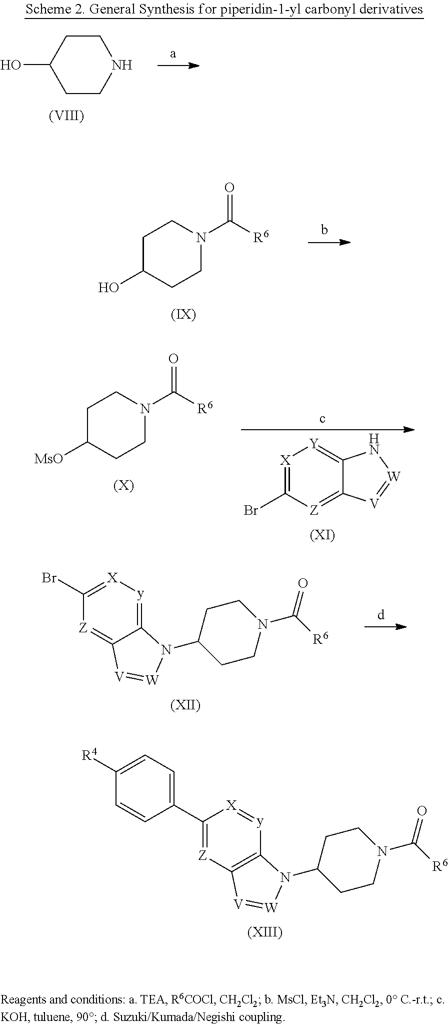

The piperidin-1-yl carbonyl-based ligands of XIII can be prepared following the general Scheme 2. Alcohol IX was prepared from piperidin-4-ol and (Boc)₂O (or other chloride compounds). And then, the following methylsulfonyl compound X, bromoheterocyclic compound XII and the final product XIII were synthesized in a similar manner as described in Scheme 1.

### Example 1

### 1-(1-(5-ethylpyrimidin-2-yl)piperidin-4-yl)-5-(4-(methylsulfonyl)phenyl)-1H-indole

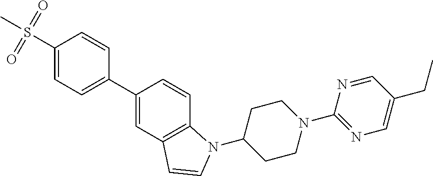 [SMILES: CCc1cnc(N2CCC(n3ccc4cc(-c5ccc(S(C)(=O)=O)cc5)ccc43)CC2)nc1]

### Step 1: 1-(5-ethylpyrimidin-2-yl)piperidin-4-ol

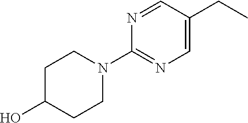

To a solution of piperidin-4-ol (2.55 g, 25.2 mmol) in MeCN (50 mL) was added 2-chloro-5-ethylpyrimidine (3.00 g, 21.0 mmol), followed by N-ethyl-N-isopropylpropan-2-amine (7 mL, 42.0 mmol) and the resulting reaction mixture was heated to 80° C. for 16 hrs. The mixture was diluted with water, extracted with EtOAc, washed with brine, dried over Na₂SO₄, filtered and concentrated in vacuo. The residue was purified with column chromatography (CH₂Cl₂: EtOAc=3:1 to 1:1) to afford the desired product (3.69 g, 85%) as a yellow solid.

¹H NMR (CDCl₃): δ 8.16 (2H, s), 4.37-4.42 (2H, m), 3.92-3.94 (1H, m), 3.24-3.30 (2H, m), 2.45 (2H, q, J=7.6 Hz), 1.92-1.98 (2H, m), 1.69 (1H, brs), 1.48-1.53 (2H, m), 1.19 (3H, t, J=7.6 Hz).

### Step 2: 1-(5-ethylpyrimidin-2-yl)piperidin-4-yl methanesulfonate

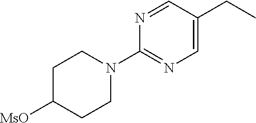 [SMILES: CCc1cnc(N2CCC(OS(C)(=O)=O)CC2)nc1]

To a solution of 1-(5-ethylpyrimidin-2-yl)piperidin-4-ol (2.42 g, 11.7 mmol) in CH₂Cl₂ (300 mL) was added Et₃N (3.24 mL, 23.4 mmol), then methanesulfonyl chloride (1 mL, 14.0 mmol) was added drop-wise at 0° C., and the resulting reaction mixture was stirred for 2 hrs at room temperature. The mixture was diluted with water, extracted with EtOAc, washed with brine, dried over Na₂SO₄, filtered and concentrated in vacuo to afford the desired product 1-(5-ethylpyrimidin-2-yl)piperidin-4-yl methanesulfonate (3.28 g, 98%) as a yellow solid.

¹H NMR (CDCl₃): δ 8.18 (2H, s), 4.95-4.99 (1H, m), 4.17-4.22 (2H, m), 3.57-3.62 (2H, m), 3.05 (3H, s), 2.47 (2H, q, J=7.6 Hz), 2.04-2.06 (2H, m), 1.87-1.90 (2H, m), 1.19 (3H, t, J=7.6 Hz).

### Step 3: 5-bromo-1-(1-(5-ethylpyrimidin-2-yl)piperidin-4-yl)-1H-indole

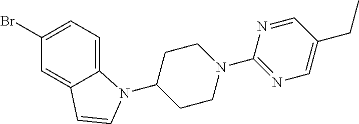 [SMILES: CCc1cnc(N2CCC(n3ccc4cc(Br)ccc43)CC2)nc1]

To a solution of 1-(5-ethylpyrimidin-2-yl)piperidin-4-yl methanesulfonate (200 mg, 1.02 mmol) in anhydrous toluene (100 mL) was added 5-bromo-1H-indole (349 mg, 1.22 mmol), followed by KOH (114 mg, 2.04 mmol). The resulting reaction mixture was heated to 90° C. and stirred for 4 hrs. After cooling, the mixture was diluted with water, extracted with EtOAc, washed with brine, dried over Na₂SO₄, filtered and concentrated in vacuo. The residue was purified by column chromatography (petrol ether: EtOAc=10:1 to 5:1) to afford the desired product 5-bromo-1-(1-(5-ethylpyrimidin-2-yl)piperidin-4-yl)-1H-indole (121 mg, 31%) as a white solid.

¹H NMR (CDCl₃): δ 8.21 (2H, s), 7.75 (1H, s), 7.30 (2H, s), 7.18 (1H, d, J=3.2 Hz), 6.45 (1H, d, J=3.2 Hz), 4.96-5.01 (2H, m), 4.42-4.50 (1H, m), 3.02-3.09 (2H, m), 2.49 (2H, q, J=7.6 Hz), 2.15-2.18 (2H, m), 1.91-2.01 (2H, m), 1.27 (3H, t, J=7.6 Hz).

### Step 4: 1-(1-(5-ethylpyrimidin-2-yl)piperidin-4-yl)-5-(4-(methyl sulfonyl)phenyl)-1H-indole

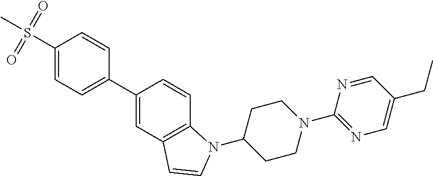 [SMILES: CCc1cnc(N2CCC(n3ccc4cc(-c5ccc(S(C)(=O)=O)cc5)ccc43)CC2)nc1]

To a solution of 5-bromo-1-(1-(5-ethylpyrimidin-2-yl) piperidin-4-yl)-1H-indole (15.0 mg, 0.04 mmol) in toluene (6 mL) was added 4,4,5,5-tetramethyl-2-(4-(methylsulfonyl)phenyl)-1,3,2-dioxaborolane (11 mg, 0.04 mmol). The resulting reaction mixture was pumped nitrogen for 30 mins, then followed Pd₂(dba)₃ (1.78 mg, 0.002 mmol), x-phos (1.85 mg, 0.004 mmol) and t-BuONa (9.35 mg, 0.1 mmol) were added and the resulting mixture was heated to 80° C. for 3 hrs under nitrogen atmosphere. After cooling, the mixture was diluted with water, extracted with EtOAc, washed with brine, dried over Na₂SO₄, filtered and concentrated in vacuo. The residue was purified by column chromatography (petrol ether: EtOAc=4:1 to 2:1) to afford the desired product 1-(1-(5-ethylpyrimidin-2-yl)piperidin-4-yl)-5-(4-(methyl sulfonyl) phenyl)-1H-indole (13.0 mg, 73%) as a white solid.

¹H NMR (CDCl₃): δ 8.23 (2H, s), 7.98-8.00 (2H, m), 7.89 (1H, d, J=1.6 Hz), 7.82-7.84 (2H, m), 7.52 (1H, d, J=8.0 Hz), 7.48 (1H, dd, J=1.6, 8.0 Hz), 7.25 (1H, d, J=3.6 Hz), 6.60 (1H, d, J=3.6 Hz), 5.00-5.03 (2H, m), 4.52-4.58 (1H, m), 3.07-3.13 (5H, m), 2.50 (2H, q, J=7.6 Hz), 2.20-2.24 (2H, m), 1.97-2.07 (2H, m), 1.22 (3H, t, J=7.6 Hz).

### Example 2

### 6-(4-(1H-tetrazol-1-yl)phenyl)-3-(1-(5-ethylpyrimidin-2-yl)piperidin-4-yl)-3H-[1,2,3]triazol o[4,5-b]pyridine

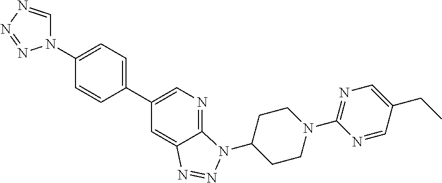 [SMILES: CCc1cnc(N2CCC(n3nnc4cc(-c5ccc(-n6cnnn6)cc5)cnc43)CC2)nc1]

### Step 1: 1-(4-bromophenyl)-1H-tetrazole

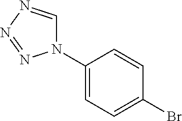 [SMILES: Brc1ccc(-n2cnnn2)cc1]

To the mixture of 4-bromoaniline (2 g, 11.6 mmol) in AcOH (12 mL), CH(OMe)₃ (1.4 g, 13.4 mmol) was dropped in. The mixture was stirred at room temperature for 1 hour. After then NaN₃ (1.25 g, 19.3 mmol) was added, and the mixture was stirred at 80° C. for 2 hours. After cooling down to room temperature, water (12 mL) and 6 N HCl solution (3.6 mL) was added, and NaNO₂ solution (0.64 g, 9.3 mmol) was dropped in under ice-bath in 5 minutes. The mixture was stirred under ice-bath for further 1 hour. After filtration and washing with water, the white flakes were dried under infra lamp to afford 1-(4-bromophenyl)-1H-tetrazole (2.20 g, 98%). ¹H NMR (CDCl₃): δ 8.98 (1H, s), 7.74 (2H, d, J=8.0 Hz), 7.61 (2H, d, J=8.0 Hz).

### Step 2: 1-(4-(4,4,5,5-tetramethyl-1,3,2-dioxaborolan-2-yl)phenyl)-1H-tetrazole

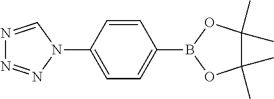 [SMILES: CC1(C)OB(c2ccc(-n3cnnn3)cc2)OC1(C)C]

The mixture of 1-(4-bromophenyl)-1H-tetrazole (1.0 g, 4.44 mmol), bis(pinacolato)diboron (3.35 g, 13.2 mmol), Pd(OAc)₂ (20 mg, 0.089 mmol), X-Phos (105 mg, 0.22 mmol) and K₃PO₄ (2.8 g, 13.2 mmol) in dioxane (80 mL) was stirred under N₂ atmosphere at 80° C. for 36 hours. After cooling down to room temperature, the mixture was filtered through a pad of Celite, and washed with EtOAc. The filtrate was concentrated, and the residue was purified with column chromatography (petrol ether: EtOAc=3:1) to give a white (0.58 g, 48%). ¹H NMR (CDCl₃): δ 9.02 (1H, s), 8.01 (2H, d, J=8.4 Hz), 7.72 (2H, d, J=8.4 Hz), 1.38 (12H, s).

### Step 3: tert-butyl 4-aminopiperidine-1-carboxylate

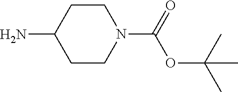

In a flask equipped with a Dean-Stark trap and condenser, a solution of piperidin-4-amine (2.00 g, 20.0 mmol) in methyl iso-butyl ketone (MIBK, 50 mL) was heated to reflux under N₂ atmosphere. After no more water was produced, the mixture was cooled to 0° C., Boc₂O (4.36 g, 20.0 mmol) dissolved in a minimum MIBK was then dropped into the flask. After stirring at room temperature for 0.5 hour, water (4 mL) was added. The aqueous layer was split off, and MIBK was evaporated in vacuo. Water and ⁱPrOH were then added, and the mixture was heated to 50° C. until completion of the hydrolysis. Solvents were then distilled off providing free primary amine (3.37 g, 85%).

¹H NMR (CDCl₃): δ 4.04 (1H, s, br), 2.85-2.75 (3H, m), 1.80-1.77 (2H, m), 1.47 (9H, s), 1.28-1.18 (2H, m).

### Step 4: 1tert-butyl 4-(5-bromo-3-nitropyridin-2-ylamino)piperidine-1-carboxylate

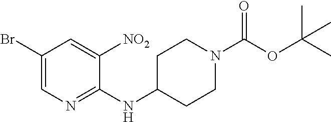 [SMILES: CC(C)(C)OC(=O)N1CCC(Nc2ncc(Br)cc2[N+](=O)[O-])CC1]

A solution of tert-butyl 4-aminopiperidine-1-carboxylate (0.84 g, 4.2 mmol), 5-bromo-2-chloro-3-nitropyridine (0.90 g, 3.8 mmol) and DIEA (1.49 g, 11.4 mmol) in NMP (15 mL) was stirred at 30° C. till the TLC showed 5-bromo-2-chloro-3-nitropyridine disappeared, and then water was added. The mixture was extracted with EtOAc, the organics were collected, washed with brine, dried over anhydrous Na₂SO₄. The solvent was concentrated, and the residue was purified with column chromatography (petrol ether: EtOAc=10:1 to 3:1) to give the desired product (1.47 g, 96%).

¹H NMR (CDCl₃): δ 8.54 (1H, d, J=2.4 Hz), 8.42 (1H, d, J=2.4 Hz), 8.11 (1H, d, J=7.6

Hz), 4.33-4.30 (1H, m), 4.11-4.06 (2H, m), 2.99 (2H, t, J=11.2 Hz), 2.06-2.04 (2H, m), 1.56-1.46 (4H, m), 1.48 (9H, s).

### Step 5: 5-bromo-3-nitro-N-(piperidin-4-yl)pyridin-2-amine

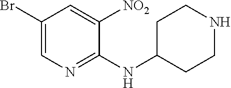 [SMILES: O=[N+]([O-])c1cc(Br)cnc1NC1CCNCC1]

The solution of tert-butyl 4-(5-bromo-3-nitropyridin-2-ylamino) piperidine-1-carboxylate (0.14 g, 0.35 mmol) in TFA (3 mL) was stirred at room temperature for 8 hours, after removal of solvents in vacuo, the residue was diluted with CH₂Cl₂, and washed with sat. Na₂CO₃ solution. The organics were collected and dried over MgSO₄, and concentrated to afford a yellow solid (0.105 g, 100%).

¹H NMR (CDCl₃): δ 8.54 (1H, dd, J=2.0 Hz, 1.2 Hz), 8.41 (1H, d, J=2.0 Hz), 8.15 (1H, d, J=5.2 Hz), 4.29-4.26 (1H, m), 3.16-3.13 (2H, m), 2.79 (2H, t, J=10.8 Hz), 2.07 (2H, d, J=9.6 Hz), 1.52-1.45 (2H, m).

### Step 6: 5-bromo-N-(1-(5-ethylpyrimidin-2-yl)piperidin-4-yl)-3-nitro pyridin-2-amine

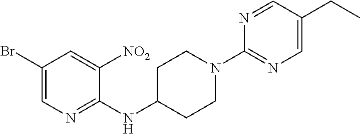 [SMILES: CCc1cnc(N2CCC(Nc3ncc(Br)cc3[N+](=O)[O-])CC2)nc1]

The mixture of 5-bromo-3-nitro-N-(piperidin-4-yl)pyridin-2-amine (105 mg, 0.349 mmol), 2-chloro-5-ethylpyrimidine (60 mg, 0.418 mmol) and K₂CO₃ (144 mg, 1.05 mmol) in DMF (5 mL) was stirred at 85° C. for 24 hours. After cooled down to room temperature, excess water was added, and the mixture was extracted with EtOAc. The organics was collected, dried over MgSO₄ and concentrated in vacuo. The residue was purified with column chromatography (petrol ether: EtOAc=1:40 to 1:20) to give a yellow solid (76 mg, 58%).

¹H NMR (CDCl₃): δ 8.55 (1H, d, J=3.2 Hz), 8.44 (1H, d, J=2.4 Hz), 8.15 (1H, d, J=8.0 Hz), 4.65 (2H, d, J=13.6 Hz), 4.49-4.41 (1H, m), 3.20 (2H, t, J=12.4 Hz), 2.48 (2H, q, J=7.6 Hz), 1.60-1.54 (2H, m), 1.20 (3H, t, J=7.6 Hz).

### Step 7: 5-(4-(1H-tetrazol-1-yl)phenyl)-N-(1-(5-ethylpyrimidin-2-yl) piperidin-4-yl)-3-nitropyridin-2-amine

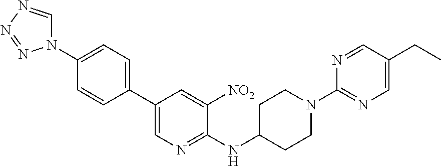 [SMILES: CCc1cnc(N2CCC(Nc3ncc(-c4ccc(-n5cnnn5)cc4)cc3[N+](=O)[O-])CC2)nc1]

The mixture of 5-bromo-N-(1-(5-ethylpyrimidin-2-yl)piperidin-4-yl)-3-nitropyridin-2-amine (35 mg, 0.092 mmol), 2-(4-(4,4,5,5-tetramethyl-1,3,2-dioxaborolan-2-yl)phenyl)-2H-tetrazole (50 mg, 0.184 mmol), PdCl₂(dppf) (1 mg, 0.00184 mmol) and K₃PO₄ (59 mg, 0.276 mmol) in dried dioxane (10 mL) was stirred at N₂ atmosphere at 88° C. for 20 hours, and then the reaction was cooled down to room temperature. After filtration and concentration, the residue was purified with column chromatography (MeOH: CH₂Cl₂=1:50) to give a yellow solid (28 mg, 64%).

¹H NMR (CDCl₃): δ 9.04 (1H, s), 8.75 (1H, d, J=2.4 Hz), 8.70 (1H, d, J=2.4 Hz), 8.30 (1H, d, J=7.6 Hz), 8.20 (2H, s), 7.84 (2H, dd, J=6.8 Hz, 2.4 Hz), 7.78 (2H, dd, J=6.8 Hz, 2.4 Hz), 4.68 (2H, d, J=13.6 Hz), 4.60-4.53 (1H, m), 3.24 (2H, t, J=11.2 Hz), 2.49 (2H, q, J=7.6 Hz), 2.20 (2H, d, J=8.8 Hz), 1.66-1.63 (2H, m), 1.21 (3H, t, J=7.6 Hz).

### Step 8: 5-(4-(1H-tetrazol-1-yl)phenyl)-N2-(1-(5-ethylpyrimidin-2-yl) piperidin-4-yl)pyridine-2,3-diamine

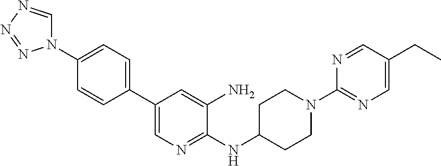 [SMILES: CCc1cnc(N2CCC(Nc3ncc(-c4ccc(-n5cnnn5)cc4)cc3N)CC2)nc1]

The mixture of 5-(4-(1H-tetrazol-1-yl)phenyl)-N-(1-(5-ethylpyrimidin-2-yl)piperidin-4-yl)-3-nitropyridin-2-amine (28 mg, 0.0592 mmol) and Pd/C (6 mg, 10%, 0.00564 mmol) in methanol was stirred under H₂ atmosphere at room temperature overnight, after filtration through a pad of Celite and concentration, a pale yellow solid (26 mg, 99%) was afford. ¹H NMR (CDCl₃): δ 9.01 (1H, s), 8.18 (2H, s), 8.06 (1H, d, J=2.0 Hz), 7.75-7.68 (4H, m), 7.13 (1H, d, J=2.0 Hz), 4.68 (2H, d, J=13.6 Hz), 4.31-4.28 (2H, m), 3.22 (2H, s, br), 3.19 (2H, t, J=13.6 Hz), 2.47 (2H, q, J=7.6 Hz), 2.23 (2H, d, J=11.2 Hz), 1.53-1.45 (2H, m), 1.21 (3H, t, J=7.6 Hz).

### Step 9: 6-(4-(1H-tetrazol-1-yl)phenyl)-3-(1-(5-ethylpyrimidin-2-yl) piperidin-4-yl)-3H-[1,2,3]triazolo[4,5-b]pyridine

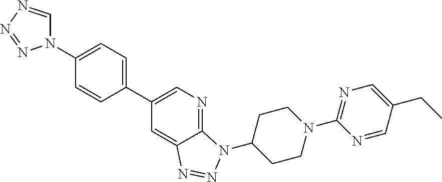 [SMILES: CCc1cnc(N2CCC(n3nnc4cc(-c5ccc(-n6cnnn6)cc5)cnc43)CC2)nc1]

To the mixture of 5-(4-(1H-tetrazol-1-yl)phenyl)-N-(1-(5-ethylpyrimidin-2-yl)piperidin-4-yl)-3-nitropyridin-2-amine (26 mg, 0.059 mmol) in HOAc-H₂O—CH₂Cl₂ (1:1:1, 3 mL), NaNO₂ (5 mg, 0.077 mmol) was added under ice-bath, then the mixture was stirred at room temperature for 1 hour. After excess CH₂Cl₂ was added, the organics were collected and washed with sat. NaHCO₃ solution, dried over MgSO₄ and concentrated to afford a grey solid (8 mg, 30%).

¹H NMR (CDCl₃): δ 9.09 (1H, s), 8.93 (1H, d, J=2.0 Hz), 8.56 (1H, d, J=2.0 Hz), 8.23 (2H, s), 7.92-7.86 (4H, m), 5.14-5.06 (2H, m), 5.00 (2H, d, J=14.0 Hz), 3.23 (2H, t, J=13.6 Hz), 2.56-2.48 (4H, m), 2.34 (2H, d, J=3.6 Hz), 1.23 (3H, t, J=7.6 Hz).

### Example 3

### 5-(4-(1H-tetrazol-1-yl)phenyl)-1-(1-(5-ethylpyrimidin-2-yl)piperidin-4-yl)-1H-indole

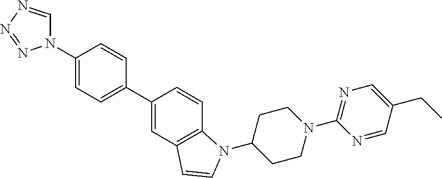 [SMILES: CCc1cnc(N2CCC(n3ccc4cc(-c5ccc(-n6cnnn6)cc5)ccc43)CC2)nc1]

5-(4-(1H-tetrazol-1-yl)phenyl)-1-(1-(5-ethylpyrimidin-2-yl)piperidin-4-yl)-1H-indole was synthesized from 1-(4-(4,4,5,5-tetramethyl-1,3,2-dioxaborolan-2-yl)phenyl)-1H-tetrazole (Example 2, Step 2) and 5-bromo-1-(1-(5-ethylpyrimidin-2-yl)piperidin-4-yl)-1H-indole (Example 1, Step 3) in a similar manner as described in Example 2, Step 7.

¹H NMR (CDCl₃) δ 9.02 (1H, s), 8.23 (2H, s), 7.76-7.91 (5H, m), 7.49-7.56 (2H, m), 6.61-6.62 (1H, m), 5.00-5.04 (2H, m), 4.53-4.60 (1H, m), 3.05-3.14 (2H, m), 2.51 (2H, q, J=7.6 Hz), 2.21-2.25 (2H, m), 1.98-2.08 (2H, m), 1.23 (3H, t, J=7.6 Hz).

### Example 4

### 1-(1-(5-ethylpyrimidin-2-yl)piperidin-4-yl)-5-(4-(methyl sulfonyl)phenyl)-1H-pyrrolo[2,3-b ]pyridine

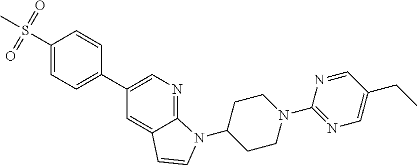 [SMILES: CCc1cnc(N2CCC(n3ccc4cc(-c5ccc(S(C)(=O)=O)cc5)cnc43)CC2)nc1]

### Step 1: 5-bromo-1-(1-(5-ethylpyrimidin-2-yl)piperidin-4-yl)-1H-pyrrolo[2,3-b]pyridine

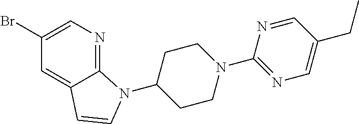 [SMILES: CCc1cnc(N2CCC(n3ccc4cc(Br)cnc43)CC2)nc1]

5-bromo-1-(1-(5-ethylpyrimidin-2-yl)piperidin-4-yl)-1H-pyrrolo[2,3-b]pyridine was synthesized from 1-(5-ethylpyrimidin-2-yl)piperidin-4-yl methanesulfonate (Example 1, Step 2) and 5-bromo-1H-pyrrolo[2,3-b]pyridine in a similar manner as described in Example 1, Step 3.

¹H NMR (CDCl₃): δ 8.33 (1H, d, J=2.0 Hz), 8.21 (2H, s), 8.02 (1H, d, J=2.0 Hz), 7.27 (1H, d, J=3.6 Hz), 6.41 (1H, d, J=3.6 Hz), 4.94-5.07 (3H, m), 3.07-3.14 (2H, m), 2.50 (2H, q, J=7.6 Hz), 2.14-2.18 (2H, m), 1.89-2.00 (2H, m), 1.21 (3H, t, J=7.6 Hz).

### Step 2: 1-(1-(5-ethylpyrimidin-2-yl)piperidin-4-yl)-5-(4-(methylsulfonyl)phenyl)-1H-pyrrolo[2,3-b]pyridine

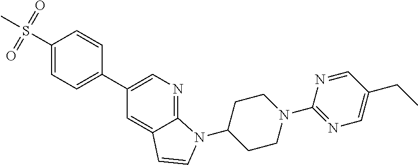 [SMILES: CCc1cnc(N2CCC(n3ccc4cc(-c5ccc(S(C)(=O)=O)cc5)cnc43)CC2)nc1]

1-(1-(5-ethylpyrimidin-2-yl)piperidin-4-yl)-5-(4-(methylsulfonyl)phenyl)-1H-pyrrolo[2,3-b ]pyridine was synthesized from 5-bromo-1-(1-(5-ethyl pyrimidin-2-yl)piperidin-4-yl)-1H-pyrrolo[2,3-b]pyridine and 4,4,5,5-tetramethyl-2-(4-(methylsulfonyl)phenyl)-1,3,2-dioxaborolane in a similar manner as described in Example 1, Step 4.

¹H NMR (CDCl₃): δ 8.57 (1H, d, J=2.4 Hz), 8.21 (2H, s), 8.12 (1H, d, J=2.0 Hz), 8.01 (2H, d, J=8.4 Hz), 7.81 (2H, d, J=8.4 Hz), 7.35 (1H, d, J=3.6 Hz), 6.55 (1H, d, J=3.6 Hz), 5.11-5.18 (1H, m), 4.97-5.01 (2H, m), 3.10-3.17 (5H, m), 2.50 (2H, q, J=7.6 Hz), 2.19-2.22 (2H, m), 1.95-2.05 (2H, m), 1.21 (3H, t, J=7.6 Hz).

### Example 5

### 1-(1-(5-ethylpyrimidin-2-yl)piperidin-4-yl)-3-methyl-5-(4-(methylsulfone)phenyl)-1H-indole

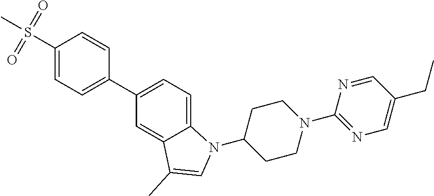 [SMILES: CCc1cnc(N2CCC(n3cc(C)c4cc(-c5ccc(S(C)(=O)=O)cc5)ccc43)CC2)nc1]

### Step 1: 5-bromo-1-(1-(5-ethylpyrimidin-2-yl)piperidin-4-yl)-3-methyl-1H-indole

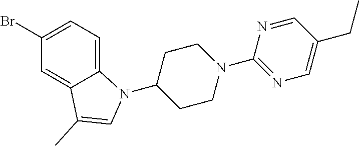 [SMILES: CCc1cnc(N2CCC(n3cc(C)c4cc(Br)ccc43)CC2)nc1]

5-bromo-1-(1-(5-ethylpyrimidin-2-yl)piperidin-4-yl)-3-methyl-1H-indole was synthesized from 1-(5-ethylpyrimidin-2-yl)piperidin-4-yl methane sulfonate (Example 1, Step 2) and 5-bromo-3-methyl-1H-indole in a similar manner as described in Example 1, Step 3.

¹H NMR (CDCl₃): δ 8.21 (2H, s), 7.68 (1H, d, J=2.0 Hz), 7.27 (1H, dd, J=8.8 Hz, 2.0

Hz), 7.23 (1H, d, J=8.8 Hz), 6.94 (1H, s), 4.93-4.98 (2H, m), 4.35-4.43 (1H, m), 2.99-3.06 (2H, m), 2.50 (2H, q, J=7.6 Hz), 2.26 (3H, s), 2.10-2.14 (2H, m), 1.87-1.97 (2H, m), 1.21 (3H, t, J=7.6 Hz).

### Step 2: 1-(1-(5-ethylpyrimidin-2-yl)piperidin-4-yl)-3-methyl-5-(4-(methylsulfonyl)-phenyl)-1H-indole

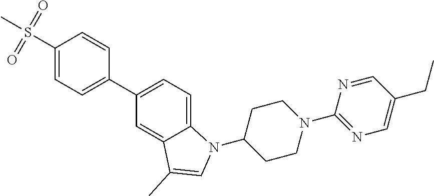 [SMILES: CCc1cnc(N2CCC(n3cc(C)c4cc(-c5ccc(S(C)(=O)=O)cc5)ccc43)CC2)nc1]

1-(1-(5-ethylpyrimidin-2-yl)piperidin-4-yl)-3-methyl-5-(4-(methyl sulfonyl)phenyl)-1H-indole was synthesized from 5-bromo-1-(1-(5-ethyl pyrimidin-2-yl)piperidin-4-yl)-3-methyl-1H-indole and 4,4,5,5-tetra methyl-2-(4-(methylsulfonyl)phenyl)-1,3,2-dioxaborolane in a similar manner as described in Example 1, Step 4.

¹H NMR (CDCl₃): δ 8.22 (2H, s), 7.99 (2H, d, J=8.4 Hz), 7.85 (2H, d, J=8.4 Hz), 7.80 (1H, s), 7.47 (1H, d, J=1.2 Hz), 7.01 (1H, d, J=0.8 Hz), 4.97-5.00 (2H, m), 4.45-4.52 (1H, m), 2.99-3.10 (5H, m), 2.50 (2H, q, J=7.6 Hz), 2.36 (3H, s), 2.14-2.18 (2H, m), 1.93-2.04 (2H, m), 1.21 (3H, t, J=7.6 Hz).

### Example 6

### 1-(1-(5-ethylpyrimidin-2-yl)piperidin-4-yl)-6-fluoro-5-(4-(methylsulfony)phenyl)-1H-indole

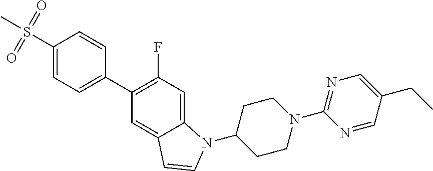 [SMILES: CCc1cnc(N2CCC(n3ccc4cc(-c5ccc(S(C)(=O)=O)cc5)c(F)cc43)CC2)nc1]

### Step 1: 4-bromo-5-fluoro-2-iodoaniline

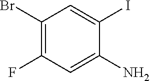

To a solution of 4-bromo-3-fluoroaniline (6.0 g, 31.5 mmol) in AcOH (100 mL) was added NIS (7.5 g, 33.3 mmol) in one portion. The reaction mixture was stirred at room temperature for 3.5 hours, basified with 2 N aqueous NaOH until pH=10. EtOAc was added and the organic layer was separated and the aqueous layer was extracted with EtOAc. The combined organic layers were washed with brine, dried over Na₂SO₄, filtered, and concentrated. The crude product (10.2 g) was used directly in the next step without further purification.

### Step 2: 4-bromo-5-fluoro-2-((trimethylsilyl)ethynyl)aniline

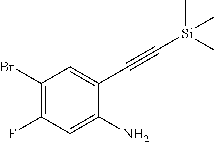

To a suspension of 4-bromo-5-fluoro-2-iodoaniline (10.2 g, 31.65 mmol), Pd(PPh₃)₂Cl₂ (1.1 g, 1.58 mmol), and CuI (0.3 g, 1.58 mmol) in Et₃N (150 mL) at 0° C. under N₂ atmosphere was dropwised trimethylsilylacetylene (6.36 mL, 37.98 mmol). The reaction mixture was warmed to ambient temperature and stirred for 2.5 hours. The reaction was concentrated, diluted with Et₂O, and filtered through Celite. The filtrate was washed with brine, dried over Na₂SO₄, filtered, and concentrated. The residue was purified with Column Chromatography (0-1% EtOAc in petrol ether) to afford the desired product (6.3 g, 70% yield) as a yellow oil.

¹H NMR (DMSO-d₆): δ 7.39 (1H, d, J=7.6 Hz), 6.64 (1H, d, J=11.6 Hz), 5.78 (2H, s), 0.23 (9H, s).

### Step 3: 5-bromo-6-fluoro-1H-indole

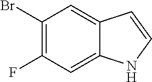 [SMILES: Fc1cc2[nH]ccc2cc1Br]

To a solution of 4-bromo-5-fluoro-2-((trimethylsilyl)ethynyl)aniline (2.3 g, 8.04 mmol) in dry DMF (5 mL) was slowly added a solution of t-BuOK (2.7 g, 24.12 mmol) in dry DMF (5 mL) at 0° C. under N₂ atmosphere. After stirring at ambient temperature overnight, the reaction mixture was heated at 80° C. for 3 hours. The reaction was diluted with EtOAc and washed with water. The aqueous layer was extracted with EtOAc and the combined organic layers were washed with brine, dried over Na₂SO₄, filtered, and concentrated. The residue was purified with Collumn Chromatography (5% EtOAc in petrol ether) to afford the desired product (0.5 g, 29% yield) as a yellow solid.

¹H NMR (CDCl₃): δ 8.22 (1H, br), 7.78 (1H, d, J=6.8 Hz), 7.20 (2H, m), 6.49 (1H, t).

### Step 4: 5-bromo-1-(1-(5-ethylpyrimidin-2-yl)piperidin-4-yl)-6-fluoro-1H-indole

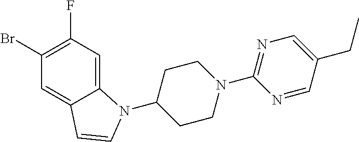 [SMILES: CCc1cnc(N2CCC(n3ccc4cc(Br)c(F)cc43)CC2)nc1]

5-bromo-1-(1-(5-ethylpyrimidin-2-yl)piperidin-4-yl)-6-fluoro-1H-indole was synthesized from 1-(5-ethylpyrimidin-2-yl)piperidin-4-yl methane sulfonate (Example 1, Step 2) and 5-bromo-6-fluoro-1H-indole in a similar manner as described in Example 1, Step 3.

¹H NMR (CDCl₃): δ 8.21 (2H, s), 7.76 (1H, dd, J=6.8 Hz, 1.6 Hz), 7.17 (2H, m), 6.44 (1H, d, J=2.4 Hz), 4.98 (2H, d, J=13.6 Hz), 4.36 (1H, m), 3.05 (2H, t, J=12.8 Hz), 2.51 (2H, q, J=7.6 Hz), 2.16 (2H, d, J=12.4 Hz), 1.97 (2H, m), 1.22 (3H, m).

### Step 5: 1-(1-(5-ethylpyrimidin-2-yl)piperidin-4-yl)-6-fluoro-5-(4-(methylsulfonyl)-phenyl)-1H-indole

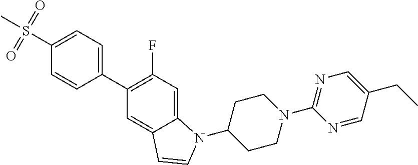 [SMILES: CCc1cnc(N2CCC(n3ccc4cc(-c5ccc(S(C)(=O)=O)cc5)c(F)cc43)CC2)nc1]

1-(1-(5-ethylpyrimidin-2-yl)piperidin-4-yl)-6-fluoro-5-(4-(methylsulfonyl)phenyl)-1H-indole was synthesized from 5-bromo-1-(1-(5-ethylpyrimidin-2-yl)piperidin-4-yl)-6-fluoro-1H-indole and 4,4,5,5-tetramethyl-2-(4-(methylsulfonyl)phenyl)-1,3,2-dioxaborolane in a similar manner as described in Example 1, Step 4.

¹H NMR (CDCl₃): δ 8.22 (2H, s), 8.0 (2H, dd, J=6.4 Hz, 1.6 Hz), 7.78 (2H, dd, J=8.4 Hz, 1.6 Hz), 7.66 (1H, d, J=7.2 Hz), 7.22 (2H, m), 6.56 (1H, d, J=3.2 Hz), 5.0 (2H, d, J=11.2 Hz), 4.43 (1H, m), 3.10 (3H, s), 3.05 (2H, m), 2.5 (2H, q, J=7.6 Hz), 2.20 (2H, d, J=10.0 Hz), 2.0 (2H, m), 1.22 (3H, t, J=7.6 Hz).

### Example 7

### tert-butyl 4-(6-fluoro-5-(4-(methylsulfonyl)phenyl)-1H-indol-1-yl)piperidine-1-carboxylate

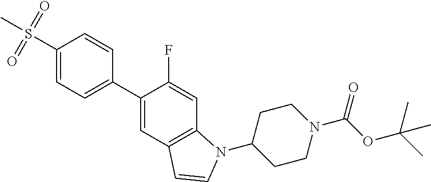 [SMILES: CC(C)(C)OC(=O)N1CCC(n2ccc3cc(-c4ccc(S(C)(=O)=O)cc4)c(F)cc32)CC1]

### Step 1: tert-butyl 4-(5-bromo-6-fluoro-1H-indol-1-yl)piperidine-1-carboxylate

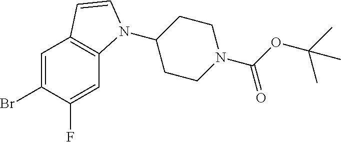 [SMILES: CC(C)(C)OC(=O)N1CCC(n2ccc3cc(Br)c(F)cc32)CC1]

tert-butyl 4-(5-bromo-6-fluoro-1H-indol-1-yl)piperidine-1-carboxylate was synthesized from 5-bromo-6-fluoro-1H-indole (Example 6, step 3) and tert-butyl 4-(methylsulfonyloxy)piperidine-1-carboxylate in a similar manner as described in Example 1, step 3.

¹H NMR (CDCl₃) δ 7.76 (1H, d, J=7.2 Hz), 7.13-7.18 (2H, m), 6.45 (1H, d, J=3.2 Hz), 4.34 (2H, br), 4.18-4.26 (1H, m), 2.91 (2H, t, J=12.0 Hz), 2.06 (2H, t, J=12.0 Hz), 1.83-1.93 (2H, m), 1.50 (9H, s).

### Step 2: tert-butyl 4-(6-fluoro-5-(4-(methylsulfonyl)phenyl)-1H-indol-1-yl)piperidine-1-carboxylate

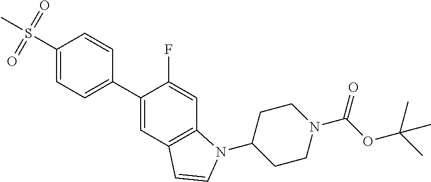 [SMILES: CC(C)(C)OC(=O)N1CCC(n2ccc3cc(-c4ccc(S(C)(=O)=O)cc4)c(F)cc32)CC1]

tert-butyl 4-(6-fluoro-5-(4-(methylsulfonyl)phenyl)-1H-indol-1-yl) piperidine-1-carboxylate was synthesized from tert-butyl 4-(5-bromo-6-fluoro-1H-indol-1-yl)piperidine-1-carboxylate (Example 7, step 1) and 4,4,5,5-tetramethyl-2-(4-(methylsulfonyl)phenyl)-1,3,2-dioxa borolane in a similar manner as described in Example 1, step 4.

¹H NMR (CDCl₃) δ 8.00-8.03 (2H, m), 7.78-7.81 (2H, m), 7.67 (1H, d, J=7.6 Hz), 7.18-7.24 (2H, m), 6.58 (1H, d, J=3.2 Hz), 4.27-4.39 (3H, m), 3.12 (3H, s), 2.92-2.98 (2H, m), 2.10-2.15 (2H, m), 1.89-2.00 (2H, m), 1.52 (9H, s).

### Example 8

### 3-chloro-1-(1-(5-ethylpyrimidin-2-yl)piperidin-4-yl)-5-(4-(methylsulfonl)phenyl)-1H-indole

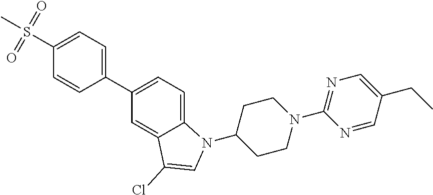 [SMILES: CCc1cnc(N2CCC(n3cc(Cl)c4cc(-c5ccc(S(C)(=O)=O)cc5)ccc43)CC2)nc1]

To a solution of 1-(1-(5-ethylpyrimidin-2-yl)piperidin-4-yl)-5-(4-(methylsulfonyl)phenyl-1H-indole (7.1 mg, 0.015 mmol) in dry DMF (1 mL) was added NCS (2.6 mg, 0.019 mmol). The reaction mixture was stirred at ambient temperature under N₂ atmosphere for 12 hours. The reaction mixture was diluted with EtOAc and washed with water. The aqueous layer was extracted with EtOAc and the combined organic layers were concentrated. The crude product was purified with Column Chromatography (EtOAc: petroleum=1:1) to give the desired product (1.5 mg, 20% yield).

¹H NMR (CDCl₃): δ 8.22 (2H, s), 8.01 (2H, d, J=8.4 Hz), 7.85 (2H, d, J=8.4 Hz), 7.52 (2H, s), 7.21 (1H, s), 5.0 (2H, d, J=13.6 Hz), 4.53 (1H, m), 3.10 (3H, s), 3.05 (2H, m), 2.5 (q, 2H, J=7.6 Hz), 2.19 (2H, d, J=10.8 Hz), 1.96 (2H, m), 1.23 (3H, t, J=7.6 Hz).

### Example 9

### 1-(1-(5-ethylpyrimidin-2-yl)piperidin-4-yl)-5-(4-(methylsulfonyl)phenyl)-1H-pyrrolo[2,3-c ]pyridine

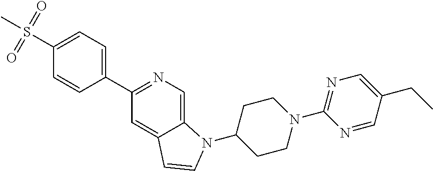 [SMILES: CCc1cnc(N2CCC(n3ccc4cc(-c5ccc(S(C)(=O)=O)cc5)ncc43)CC2)nc1]

### Step 1: 4-methyl-5-nitropyridin-2-amine

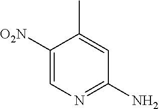

To the solution of 4-methylpyridin-2-amine (5.80 g, 53.6 mmol) in con. H₂SO₄ (8 mL), the mixture of sulfuric acid (4.00 mL, 75 mmol) and nitric acid (4.05 mL, 91 mmol) was added at 5-20° C. during 15 minutes. The mixture was stirred at room temperature for 30 minutes, and then heated to 35-40° C. for 2 hours, 50° C. for 5 hours. The mixture was poured onto ice, adjusted pH to 9 with con. NH₄OH. The precipitates were collected and purified with Column Chromatography (EtOAc: petrol ether=1:3) to give the desired product (1.5 g, 18%).

¹H NMR (DMSO-d₆): δ 8.75 (1H, s), 7.27 (2H, s), 6.31 (1H, s), 2.49 (3H, s).

### Step 2: 2-bromo-4-methyl-5-nitropyridine

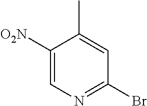

To the mixture of tert-butyl nitrite (202 mg, 1.96 mmol) and CuBr (225 mg, 1.57 mmol) in CH₃CN (2 mL), 4-methyl-5-nitropyridin-2-amine (200 mg, 1.31 mmol) was added portion wise at 60-65° C. The mixture was heated to 70° C. for 2 hours, and then cooled and concentrated. The residue was poured into EtOAc, washed with water, brine, dried over Na₂SO₄. The organic solvent was concentrated in vacuo and purified with Column Chromatography (petrol ether: EtOAc=10:1) to give the desired product (50 mg, 17%).

¹H NMR (CDCl₃): δ 8.94 (1H, s), 7.53 (1H, s), 2.64 (3H, s).

### Step 3: 2-(2-bromo-5-nitropyridin-4-yl)-N,N-dimethylethenamine

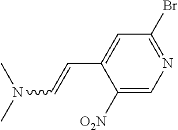

The mixture of 1,1-dimethoxy-N,N-dimethylmethanamine (27.5 mg, 0.23 mmol) and 2-bromo-4-methyl-5-nitropyridine (50 mg, 0.23 mmol) in DMF (2 mL) was heated to 100° C. and stirred for 1 hour. The solvent was concentrated to give the desired product (62 mg, 99%).

¹H NMR (CDCl₃): δ 8.74 (1H, s), 7.41 (1H, s), 7.32 (1H, d, J=12.8 Hz, 2.0 Hz), 5.93 (1H, d, J=12.8 Hz), 3.06 (6H, br).

### Step 4: 5-bromo-1H-pyrrolo[2,3-c]pyridine

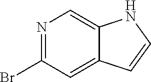

To the solution of 2-(2-bromo-5-nitropyridin-4-yl)-N,N-dimethylethenamine (62 mg, 0.23 mmol) in AcOH (2 mL), iron (127 mg, 2.28 mmol) was added at room temperature and the mixture was heated at 70° C. for 2 hours. The mixture was cooled, poured into EtOAc (30 mL), filtered through Celite. The filtration was washed with 5% NaHCO₃ solution, water and brine, dried over Na₂SO₄. The solvent was concentrated in vacuo to the desired product (25 mg, 56%).

¹H NMR (CDCl₃): δ 9.42 (1H, br), 8.60 (1H, s), 7.74 (1H, s), 7.47 (1H, d, J=2.8 Hz), 6.54 (1H, d, J=2.8 Hz).

### Step 5: 5-bromo-1-(1-(5-ethylpyrimidin-2-yl)piperidin-4-yl)-1H-pyrrolo[2,3-c]pyridine

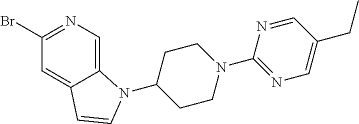 [SMILES: CCc1cnc(N2CCC(n3ccc4cc(Br)ncc43)CC2)nc1]

5-bromo-1-(1-(5-ethylpyrimidin-2-yl)piperidin-4-yl)-1H-pyrrolo[2,3-c]pyridine was synthesized from 1-(5-ethylpyrimidin-2-yl)piperidin-4-yl methanesulfonate (Example 1, Step 2) and 5-bromo-1H-pyrrolo[2,3-c]pyridine in a similar manner as described in Example 1, Step 3.

¹H NMR (CDCl₃): δ 8.60 (1H, s), 8.21 (2H, s), 7.69 (1H, s), 7.34 (1H, d, J=3.2 Hz), 6.46 (1H, d, J=3.2 Hz), 4.97-5.02 (2H, m), 4.45-4.61 (1H, m), 3.05-3.12 (2H, m), 2.50 (2H, q, J=7.6 Hz), 2.19-2.22 (2H, m), 1.95-2.05 (2H, m), 1.21 (3H, t, J=7.6 Hz).

### Step 6: 1-(1-(5-ethylpyrimidin-2-yl)piperidin-4-yl)-5-(4-(methylsulfonyl)phenyl)-1H-pyrrolo[2,3-c]pyridine

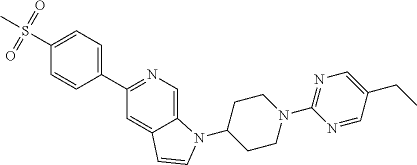 [SMILES: CCc1cnc(N2CCC(n3ccc4cc(-c5ccc(S(C)(=O)=O)cc5)ncc43)CC2)nc1]

1-(1-(5-ethylpyrimidin-2-yl)piperidin-4-yl)-5-(4-(methylsulfonyl)phenyl)-1H-pyrrolo[2,3-c ]pyridine was synthesized from 5-bromo-1-(1-(5-ethylpyrimidin-2-yl)piperidin-4-yl)-1H-pyrrolo[2,3-c]pyridine and 4,4,5,5-tetramethyl-2-(4-(methylsulfonyl)phenyl)-1,3,2-dioxaborolane in a similar manner as described in Example 1, Step 4.

¹H NMR (CDCl₃): δ 8.95 (1H, s), 8.22-8.25 (3H, m), 8.01-8.05 (3H, m), 7.38 (1H, d, J=3.2 Hz), 6.61 (1H, d, J=3.2 Hz), 5.00-5.04 (2H, m), 4.60-5.68 (1H, m), 3.10-3.15 (5H, m), 2.50 (2H, q, J=7.6 Hz), 2.24-2.28 (2H, m), 2.03-2.08 (2H, m), 1.21 (3H, t, J=7.6 Hz).

### Example 10

### tert-butyl 4-(5-(4-(methylsulfonyl)phenyl)-1H-indol-1-yl)piperidine-1-carboxylate

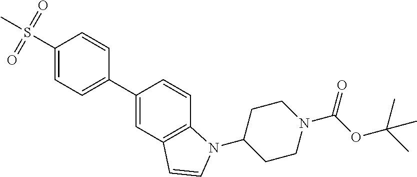 [SMILES: CC(C)(C)OC(=O)N1CCC(n2ccc3cc(-c4ccc(S(C)(=O)=O)cc4)ccc32)CC1]

### Step 1: tert-butyl 4-(5-bromoindolin-1-yl)piperidine-1-carboxylate

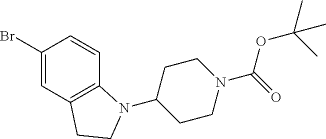 [SMILES: CC(C)(C)OC(=O)N1CCC(N2CCc3cc(Br)ccc32)CC1]

To a solution of 5-bromoindoline (50 mg, 0.25 mmol) in HOAc (3 mL) was added tert-butyl 4-oxopiperidine-1-carboxylate (55 mg, 0.28 mmol) and the resulting mixture was stirred at room temperature for 30 minutes, then NaBH(OAc)₃ (80 mg, 0.38 mmol) was added and stirred for further 1 hour. The mixture was neutralized to pH=8 with saturated aqueous NaHCO₃ and extracted with EtOAc. The combined extract was washed with brine, dried and concentrated to afford crude product which was used in the next step without further purification.

### Step 2: tert-butyl 4-(5-bromo-1H-indol-1-yl)piperidine-1-carboxylate

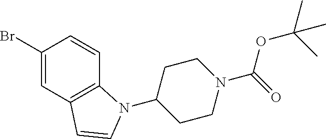 [SMILES: CC(C)(C)OC(=O)N1CCC(n2ccc3cc(Br)ccc32)CC1]

To a solution of tert-butyl 4-(5-bromoindolin-1-yl)piperidine-1-carboxylate (100 mg, 0.26 mmol) in THF (10 mL) was added 4,5-dichloro-3,6-dioxocyclohexane-1,2-dicarbonitrile (67 mg, 0.29 mmol) at 0° C. and the resulting mixture was stirred at 0° C. for 1 hour. The mixture was neutralized to pH=10 with aqueous NaOH and extracted with EtOAc. The combined extracts were washed with brine, dried and concentrated. The residue was purified with column chromatography (petrol ether: EtOAc=5:1 to 4:1) to afford desired product (84 mg, 87% in two steps).

¹H NMR (CDCl₃): δ 7.75 (1H, s), 7.24-7.28 (2H, m), 7.18 (1H, d, J=3.2 Hz), 6.46 (1H, d, J=3.2 Hz), 4.23-4.40 (3H, m), 2.84-2.98 (2H, m), 2.03-2.10 (2H, m), 1.87-1.98 (2H, m), 1.49 (9H, s).

### Step 3: tert-butyl 4-(5-(4-(methylsulfonyl)phenyl)-1H-indol-1-yl) piperidine-1-carboxylate

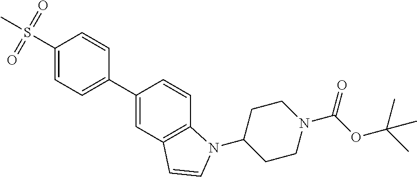 [SMILES: CC(C)(C)OC(=O)N1CCC(n2ccc3cc(-c4ccc(S(C)(=O)=O)cc4)ccc32)CC1]

tert-butyl 4-(5-(4-(methylsulfonyl)phenyl)-1H-indol-1-yl)piperidine-1-carboxylate was synthesized from tert-butyl 4-(5-bromo-1H-indol-1-yl) piperidine-1-carboxylate (Example 10, step 2) and 4,4,5,5-tetramethyl-2-(4-(methylsulfonyl)phenyl)-1,3,2-dioxaborolane in a similar manner as described in Example 1, step 4.

¹H NMR (CDCl₃): δ 7.98-8.00 (2H, m), 7.89 (1H, d, J=1.6 Hz), 7.82-7.84 (2H, m), 7.52 (1H, d, J=8.0 Hz), 7.48 (1H, dd, J=1.6, 8.0 Hz), 7.25 (1H, d, J=3.6 Hz), 6.60 (1H, d, J=3.6 Hz), 4.38-4.40 (3H, m), 3.10 (3H, s), 2.95-2.99 (2H, m), 2.05-2.13 (2H, m), 1.90-2.01 (2H, m), 1.36 (9H, s).

### Example 11

### 1-(1-(5-ethylpyrimidin-2-yl)piperidin-4-yl)-5-(2-fluoro-4-(methylsulfony)phenyl)-1H-indole

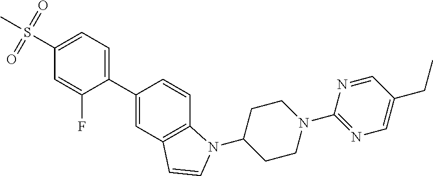 [SMILES: CCc1cnc(N2CCC(n3ccc4cc(-c5ccc(S(C)(=O)=O)cc5F)ccc43)CC2)nc1]

### Step 1: tert-butyl 5-bromoindoline-1-carboxylate

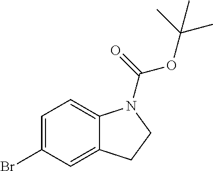 [SMILES: CC(C)(C)OC(=O)N1CCc2cc(Br)ccc21]

To a solution of 5-bromoindoline (280 mg, 1.41 mmol) in THF (15 mL) and H₂O (5 mL) was added k₂CO₃ (293 mg, 2.12 mmol), followed by di-tert-butyl dicarbonate (617 mg, 2.82 mmol) and the resulting mixture was stirred at room temperature for 2 hours. The mixture was diluted with water and extracted with EtOAc. The combined extract was washed with brine, dried and concentrated to afford desired product (338 mg, 80%). ¹H NMR (DMSO-d₆): δ 7.59 (1H, br), 7.38 (1H, s), 7.30-7.32 (2H, m), 3.90 (2H, t, J=8.8 Hz), 3.07 (2H, t, J=8.8 Hz), 1.50 (9H, s).

### Step 2: tert-butyl 5-(4,4,5,5-tetramethyl-1,3,2-dioxaborolan-2-yl)indoline-1-carboxylate

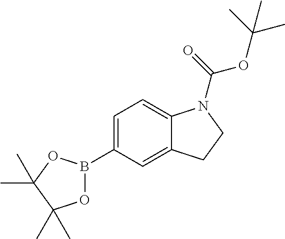 [SMILES: CC(C)(C)OC(=O)N1CCc2cc(B3OC(C)(C)C(C)(C)O3)ccc21]

To a solution of tert-butyl 5-bromoindoline-1-carboxylate (50 mg, 0.17 mmol) in DMF (5 mL) was added 4,4,4′,4′,5,5,5′,5′-octamethyl-2,2′-bi (1,3,2-dioxaborolane) (63.9 mg, 0.25 mmol) and the mixture was pumped with nitrogen for 30 minutes, then PdCl₂(dppf) (12.3 mg, 0.017 mmol) and KOAc (41.1 mg, 0.42 mmol) were added to the mixture and stirred at 90° C. for 15 hours under nitrogen atmosphere. The mixture was diluted with water and extracted with EtOAc. The combined extract was washed with brine, dried and concentrated in vacuo. The residue was purified with column chromatography (petrol ether: EtOAc=50:1 to 30:1) to afford the desired product (36.5 mg, 63%).

¹H NMR (CDCl₃): δ 7.78 (1H, br), 7.63-7.67 (2H, m), 7.61 (1H, s), 3.97 (2H, t, J=8.8 Hz), 3.09 (2H, t, J=8.8 Hz), 1.58 (9H, s), 1.35 (12H, s).

### Step 3: 5-(4,4,5,5-tetramethyl-1,3,2-dioxaborolan-2-yl)indoline

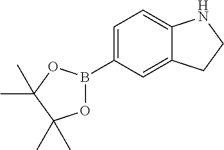 [SMILES: CC1(C)OB(c2ccc3c(c2)CCN3)OC1(C)C]

To the solution of tert-butyl 5-(4,4,5,5-tetramethyl-1,3,2-dioxaborolan-2-yl)indoline-1-carboxylate (120 mg, 0.35 mmol) in CH₂Cl₂ (5 mL) was added 2,2,2-trifluoroacetic acid (1 mL) and the resulting mixture was stirred at room temperature for 2 hours. The mixture was neutralized to pH=8 with saturated aqueous NaHCO₃ and extracted with CH₂Cl₂. The combined extracts were washed with brine, dried and concentrated in vacuo to afford the desired product (74 mg, 87%).

¹H NMR (CDCl₃): δ 7.56 (1H, s), 7.50 (1H, d, J=8.0 Hz), 6.60 (1H, d, J=8.0 Hz), 3.57 (2H, t, J=8.8 Hz), 3.02 (2H, t, J=8.8 Hz), 1.32 (12H, s).

### Step 4: tert-butyl 4-(5-(4,4,5,5-tetramethyl-1,3,2-dioxaborolan-2-yl) indolin-1-yl)piperidine-1-carboxylate

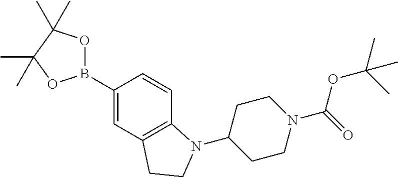 [SMILES: CC(C)(C)OC(=O)N1CCC(N2CCc3cc(B4OC(C)(C)C(C)(C)O4)ccc32)CC1]

To a solution of 5-(4,4,5,5-tetramethyl-1,3,2-dioxaborolan-2-yl)indoline (74 mg, 0.30 mmol) in HOAc (4 mL) was added tert-butyl 4-oxopiperidine-1-carboxylate (66 mg, 0.33 mmol) and the resulting mixture was stirred at room temperature for 30 minutes, then NaBH(OAc)₃ (96 mg, 0.45 mmol) was added and the mixture was stirred for further 1 hour. The mixture was neutralized to pH=8 with saturated aqueous NaHCO₃ and extracted with EtOAc. The combined extracts was washed with brine, dried and concentrated in vacuo to afford crude product which was used in the next step without further purification.

### Step 5: tert-butyl 4-(5-(4,4,5,5-tetramethyl-1,3,2-dioxaborolan-2-yl)-1H-indol-1-yl)piperidine-1-carboxylate

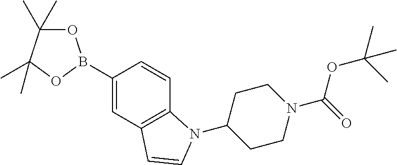 [SMILES: CC(C)(C)OC(=O)N1CCC(n2ccc3cc(B4OC(C)(C)C(C)(C)O4)ccc32)CC1]

To a solution of tert-butyl 4-(5-(4,4,5,5-tetramethyl-1,3,2-dioxaborolan-2-yl)indolin-1-yl)piperidine-1-carboxylate (13.0 mg, 0.30 mmol) in THF (10 mL) was added 4,5-dichloro-3,6-dioxocyclohexane-1,2-dicarbonitrile (77 mg, 0.33 mmol) at 0° C. and the resulting mixture was stirred at 0° C. for 1 hour. The mixture was neutralized to pH=10 with aqueous NaOH and extracted with EtOAc. The combined extract was washed with brine, dried and concentrated in vacuo. The residue was purified with column chromatography (petrol ether: EtOAc=3:1 to 2:1) to afford the desired product (78 mg, 60% in two steps).

¹H NMR (CDCl₃): δ 8.16 (1H, s), 7.65 (1H, d, J=8.4 Hz), 7.36 (1H, d, J=8.8 Hz), 7.17 (1H, d, J=3.2 Hz), 6.54 (1H, d, J=3.2 Hz), 4.29-4.42 (3H, m), 2.89-2.96 (2H, m), 2.06-2.09 (2H, m), 1.89-1.95 (2H, m), 1.50 (9H, s), 1.36 (12H, s).

### Step 6: tert-butyl 4-(5-(2-fluoro-4-(methylsulfonyl)phenyl)-1H-indol-1-yl)piperidine-1-carboxylate

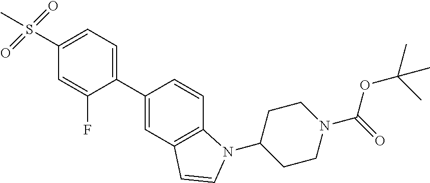 [SMILES: CC(C)(C)OC(=O)N1CCC(n2ccc3cc(-c4ccc(S(C)(=O)=O)cc4F)ccc32)CC1]

tert-butyl 4-(5-(2-fluoro-4-(methylsulfonyl)phenyl)-1H-indol-1-yl) piperidine-1-carboxylate was synthesized from tert-butyl 4-(5-(4,4,5,5-tetramethyl-1,3,2-dioxaborolan-2-yl)-1H-indol-1-yl)piperidine-1-carboxylate (Example 11, step 5) and 1-bromo-2-fluoro-4-(methylsulfonyl) benzene in a similar manner as described in Example 1, step 4.

¹H NMR (CDCl₃): δ 7.83 (1H, s), 7.74-7.79 (1H, m), 7.62-7.72 (2H, m), 7.45-7.50 (1H, m), 7.39-7.42 (1H, m), 7.25 (1H, d, J=3.2 Hz), 6.60 (1H, d, J=3.2 Hz), 4.31-4.43 (3H, m), 3.10 (3H, s), 2.89-2.97 (2H, m), 2.09-2.12 (2H, m), 1.91-1.99 (2H, m), 1.19 (9H, s).

### Step 7: 5-(2-fluoro-4-(methylsulfonyl)phenyl)-1-(piperidin-4-yl)-1H-indole

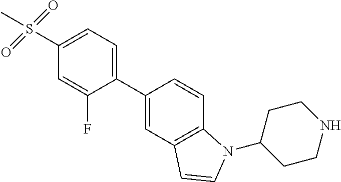 [SMILES: CS(=O)(=O)c1ccc(-c2ccc3c(ccn3C3CCNCC3)c2)c(F)c1]

To a solution of tert-butyl 4-(5-(2-fluoro-4-(methylsulfonyl)phenyl)-1H-indol-1-yl)piperidine-1-carboxylate (23 mg, 0.05 mmol) in CH₂Cl₂ (5 mL) was added 2,2,2-trifluoroacetic acid (0.3 mL) and the resulting mixture was stirred at room temperature for 2 hours. The mixture was neutralized with aqueous saturated NaHCO₃ to pH=8 and extracted with CH₂Cl₂. The combined extract was washed with brine, dried and concentrated in vacuo to afford the desired product (16 mg, 90%).

¹H NMR (CDCl₃): δ 7.85 (1H, s), 7.77-7.80 (1H, m), 7.69-7.75 (2H, m), 7.49-7.51 (1H, m), 7.40-7.42 (1H, m), 7.31 (1H, d, J=3.2 Hz), 6.60 (1H, d, J=3.2 Hz), 4.29-4.40 (2H, m), 3.29-3.32 (2H, m), 3.12 (3H, s), 2.93-2.90 (2H, m), 1.96-2.02 (2H, m).

### Step 8: 1-(1-(5-ethylpyrimidin-2-yl)piperidin-4-yl)-5-(2-fluoro-4-(methylsulfonyl)phenyl)-1H-indole

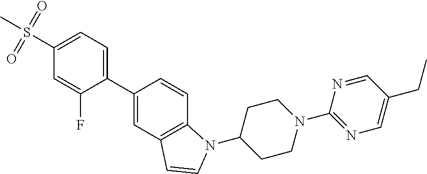 [SMILES: CCc1cnc(N2CCC(n3ccc4cc(-c5ccc(S(C)(=O)=O)cc5F)ccc43)CC2)nc1]

To a solution of 5-(2-fluoro-4-(methylsulfonyl)phenyl)-1-(piperidin-4-yl)-1H-indole (16 mg, 0.04 mmol) in CH₃CN (5 mL) was added DIEA (11 mg, 0.08 mmol), followed by 2-chloro-5-ethylpyrimidine (9.19 mg, 0.06 mmol) and the resulting mixture was stirred at 90° C. for 24 hours. The mixture was diluted with water and extracted with EtOAc. The combined extracts was washed with brine, dried and concentrated in vacuo. The residue was purified with column chromatography (petrol ether: EtOAc=3:1 to 2:1) to afford the desired product (3.8 mg, 18%).

¹H NMR (CDCl₃): δ 8.24 (2H, s), 7.87 (1H, s), 7.77-7.82 (1H, m), 7.72-7.75 (2H, m), 7.54-7.56 (1H, m), 7.44-7.46 (1H, m), 7.28 (1H, d, J=3.2 Hz), 6.62 (1H, d, J=3.2 Hz), 5.00-5.04 (2H, m), 4.53-4.61 (1H, m), 3.14 (3H, s), 3.07-3.13 (2H, m), 2.52 (2H, q, J=7.6 Hz), 2.21-2.26 (2H, m), 2.01-2.05 (2H, m), 1.26 (3H, t, J=7.6 Hz).

### Example 12

### 5-(4-(1H-tetrazol-1-yl)phenyl)-1-(1-(5-ethylpyrimidin-2-yl)piperidin-4-yl)-6-fluoro-1H-indole

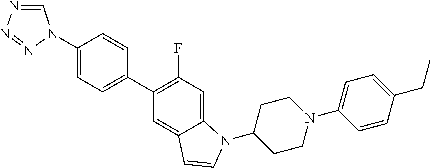 [SMILES: CCc1ccc(N2CCC(n3ccc4cc(-c5ccc(-n6cnnn6)cc5)c(F)cc43)CC2)cc1]

5-(4-(1H-tetrazol-1-yl)phenyl)-1-(1-(5-ethylpyrimidin-2-yl)piperidin-4-yl)-6-fluoro-1H-indole was synthesized from 5-bromo-1-(1-(5-ethyl pyrimidin-2-yl)piperidin-4-yl)-6-fluoro-1H-indole (Example 6, step 4) and 1-(4-(4,4,5,5-tetramethyl-1,3,2-dioxaborolan-2-yl)phenyl)-1H-tetrazole (Example 2, step 2) in a similar manner as described in Example 2, step 7.

¹H NMR (CDCl₃): δ 9.03 (1H, s), 8.23 (1H, s), 7.79 (3H, m), 7.67 (1H, d, J=7.6 Hz), 7.22 (1H, m), 6.57 (1H, s), 5.02 (2H, d, J=9.2 Hz), 4.43 (1H, m), 3.08 (1H, t, J=12.0 Hz), 2.51 (2H, m), 2.21 (3H, m), 1.22 (3H, t, J=7.6 Hz).

### Example 13

### 4-(1-(1-(5-ethylpyrimidin-2-yl)piperidin-4-yl)-6-fluoro-1H-indol-5-yl)-2-fluorobenzonitrile

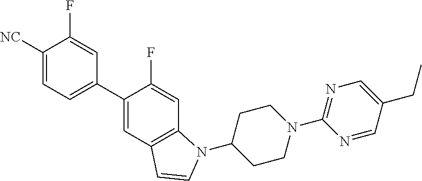 [SMILES: CCc1cnc(N2CCC(n3ccc4cc(-c5ccc(C#N)c(F)c5)c(F)cc43)CC2)nc1]

4-(1-(1-(5-ethylpyrimidin-2-yl)piperidin-4-yl)-6-fluoro-1H-indol-5-yl)-2-fluorobenzonitrile was synthesized from 5-bromo-1-(1-(5-ethyl pyrimidin-2-yl)piperidin-4-yl)-6-fluoro-1H-indole (Example 6, step 4) and 2-fluoro-4-(4,4,5,5-tetramethyl-1,3,2-dioxaborolan-2-yl)benzonitrile in a similar manner as described in Example 1, step 4.

¹H NMR (CDCl₃): δ 8.22 (2H, s), 7.67 (2H, m), 7.48 (2H, m), 7.22 (2H, m), 6.56 (1H, s), 5.00 (2H, m), 4.99-5.03 (2H, m), 4.42 (1H, m), 3.07 (2H, t, J=12.4 Hz), 2.50 (2H, m), 2.19 (2H, m), 1.99 (2H, m), 1.22 (3H, t, J=7.6 Hz).

### Example 14

### 4-(1-(1-(5-ethylpyrimidin-2-yl)piperidin-4-yl)-6-fluoro-1H-indol-5-yl)benzonitrile

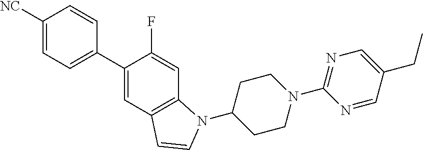 [SMILES: CCc1cnc(N2CCC(n3ccc4cc(-c5ccc(C#N)cc5)c(F)cc43)CC2)nc1]

4-(1-(1-(5-ethylpyrimidin-2-yl)piperidin-4-yl)-6-fluoro-1H-indol-5-yl)benzonitrile was synthesized from 5-bromo-1-(1-(5-ethyl pyrimidin-2-yl)piperidin-4-yl)-6-fluoro-1H-indole (Example 6, step 4) and 4-(4,4,5,5-tetramethyl-1,3,2-dioxaborolan-2-yl)benzonitrile in a similar manner as described in Example 1, step 4.

¹H NMR (CDCl₃): δ 8.22 (2H, s), 7.68-7.73 (4H, m), 7.64 (1H, d, J=7.6 Hz), 7.21-7.24 (2H, m), 6.55 (1H, m), 4.98-5.02 (2H, m), 4.43 (1H, s), 3.07 (2H, t, J=12.8 Hz), 2.51 (2H, m), 2.19 (2H, m), 1.97-2.05 (2H, m), 1.22 (3H, t, J=7.6 Hz).

### Example 15

### N-(4-(1-(1-(5-ethylpyrimidin-2-yl)piperidin-4-yl)-6-fluoro-1H-indol-5-yl)phenyl)methanesulfonamide

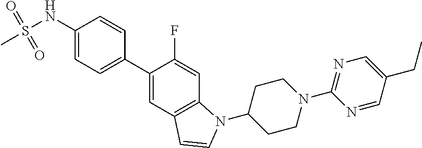 [SMILES: CCc1cnc(N2CCC(n3ccc4cc(-c5ccc(NS(C)(=O)=O)cc5)c(F)cc43)CC2)nc1]

N-(4-(1-(1-(5-ethylpyrimidin-2-yl)piperidin-4-yl)-6-fluoro-1H-indol-5-yl)phenyl)methanesulfonamide was synthesized from 5-bromo-1-(1-(5-ethyl pyrimidin-2-yl)piperidin-4-yl)-6-fluoro-1H-indole (Example 6, step 4) and N-(4-(4,4,5,5-tetramethyl-1,3,2-dioxaborolan-2-yl)phenyl)methane sulfonamide in a similar manner as described in Example 1, step 4.

¹H NMR (CDCl₃): δ 8.22 (2H, s), 7.69 (3H, m), 7.29 (2H, d, J=8.4 Hz), 7.20 (2H, m), 6.53 (1H, s), 6.42 (1H, br), 5.00 (2H, m), 4.42 (1H, m), 3.05 (5H, m), 2.51 (2H, m), 2.20 (2H, m), 1.98 (2H, m), 1.21 (3H, m).

### Example 16

### tert-butyl 4-(5-(4-(methylsulfonyl)phenyl)-1H-indazol-1-yl)piperidine-1-carboxylate

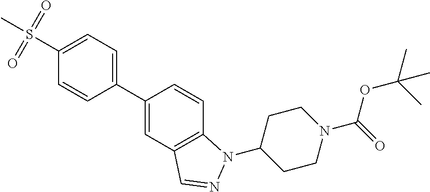 [SMILES: CC(C)(C)OC(=O)N1CCC(n2ncc3cc(-c4ccc(S(C)(=O)=O)cc4)ccc32)CC1]

### Step 1: 5-bromo-1H-indazole

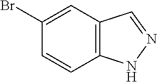

The solution of 5-bromo-2-fluorobenzaldehyde (406 mg, 2.0 mmol) in anhydrous hydrazine (10 mL) was heated at 100° C. for 24 hours, then cooled to room temperature. After removal of the residual anhydrous hydrazine under reduced pressure, the residue was separated between EtOAc and water. The organic phase was washed with brine, dried over anhydrous Na₂SO₄, concentrated under reduced pressure and purified with column chromatography (EtOAc: petrol ether=1:3) to give the desired product (154 mg, 39%).

¹H NMR (CDCl₃): δ 10.14 (1H, br), 8.03 (1H, d, J=0.8 Hz), 7.92 (1H, dd, J=0.8 Hz, J=1.6 Hz), 7.48 (1H, dd, J=1.6 Hz, J=8.8 Hz), 7.40 (1H, d, J=8.8 Hz).

### Step 2: tert-butyl 4-(5-bromo-1H-indazol-1-yl)piperidine-1-carboxylate

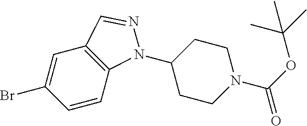 [SMILES: CC(C)(C)OC(=O)N1CCC(n2ncc3cc(Br)ccc32)CC1]

At 0° C., to a mixture of 5-bromo-1H-indazole (197 mg, 1.0 mmol) and NaH (44 mg, 1.1 mmol, 60%) in dry DMF (20 mL) was added tert-butyl 4-(methylsulfonyloxy)piperidine-1-carboxylate (307 mg, 1.1 mmol), then the reaction mixture was heated at 100° C. for 14 hours. After removal of the solvent under reduced pressure, the residue was separated between CH₂Cl₂ and water. The organic phase was washed with brine, dried over anhydrous Na₂SO₄, concentrated under reduced pressure and purified with flash column chromatography (EtOAc: petrol ether=1:4) to give tert-butyl 4-(5-bromo-1H-indazol-1-yl)piperidine-1-carboxylate (220 mg, 58%).

¹H NMR (CDCl₃): δ 7.93 (1H, d, J=0.8 Hz), 7.88 (1H, dd, J=0.8 Hz, J=1.6 Hz), 7.45 (1H, dd, J=1.6 Hz, J=8.8 Hz), 7.33 (1H, d, J=8.8 Hz), 4.53-4.55 (1H, m), 4.29-4.32 (2H, m), 2.93-2.97 (2H, m), 2.18-2.23 (2H, m), 2.00-2.05 (2H, m), 1.48 (9H, s).

### Step 3: tert-butyl 4-(5-(4-(methylsulfonyl)phenyl)-1H-indazol-1-yl)piperidine-1-carboxylate

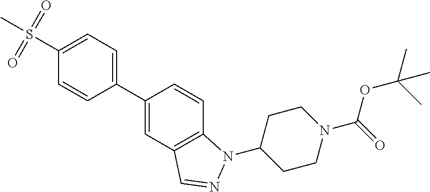 [SMILES: CC(C)(C)OC(=O)N1CCC(n2ncc3cc(-c4ccc(S(C)(=O)=O)cc4)ccc32)CC1]

tert-butyl 4-(5-(4-(methylsulfonyl)phenyl)-1H-indazol-1-yl)piperidine-1-carboxylate was synthesized from tert-butyl 4-(5-bromo-1H-indazol-1-yl)piperidine-1-carboxylate (Example 16, step 2) and 4,4,5,5-tetramethyl-2-(4-(methylsulfonyl)phenyl)-1,3,2-dioxaborolane in a similar manner as described in Example 1, step 4. ¹H NMR (CDCl₃): δ 8.10 (1H, s), 8.04 (2H, d, J=8.8 Hz), 7.98 (1H, s), 7.82 (2H, d, J=8.8 Hz), 7.64 (1H, d, J=8.8 Hz), 7.56 (1H, d, J=8.8 Hz), 4.57-4.62 (1H, m), 4.25-4.35 (2H, m), 3.11 (3H, s), 2.93-3.04 (2H, m), 2.18-2.27 (2H, m), 2.00-2.07 (2H, m), 1.48 (9H, s).

### Example 17

### 5-(4-(1H-tetrazol-1-yl)phenyl)-1-(1-(5-ethylpyrimidin-2-yl)piperidin-4-yl)-6-fluoro-1H-indazole

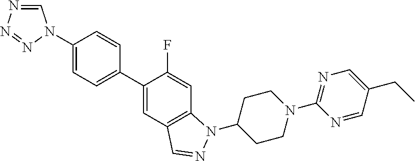 [SMILES: CCc1cnc(N2CCC(n3ncc4cc(-c5ccc(-n6cnnn6)cc5)c(F)cc43)CC2)nc1]

### Step 1: N-(5-bromo-4-fluoro-2-methylphenyl)acetamide

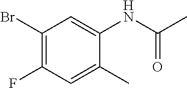

The solution of 4-fluoro-2-methylaniline (1.25 g, 10 mmol) and acetic anhydride (1.12 g, 11 mmol) in toluene (20 mL) was refluxed for 1 hour, and then cooled to room temperature. The colorless solid precipitated out which was filtered, washed with petro ether, taken in HOAc (15 mL) and treated dropwise with a solution of bromine (1.60 g, 10 mmol) in HOAc (5 mL). The reaction mixture was stirred at room temperature overnight, and then quenched with water (5 mL). The solid was filtered, washed with petro ether and dried in vacuo to give the desired product (1.97 g, 80%).

¹H NMR (CDCl₃): δ 7.92 (1H, d, J=7.6 Hz), 7.33 (1H, d, J=7.6 Hz), 6.91 (1H, br), 2.22 (6H, s).

### Step 2: 1-(5-bromo-6-fluoro-1H-indazol-1-yl)ethanone

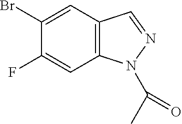

The mixture of N-(5-bromo-4-fluoro-2-methylphenyl)acetamide (1.97 g, 8.0 mmol), acetic anhydride (2.45 g, 24.0 mmol), potassium acetate (1.57 g, 16.0 mmol), isoamy nitrite (2.08 g, 16.0 mmol) and 18-crown-6 (106 mg, 0.4 mmol) in CHCl₃ (50 mL) was heated at 65° C. overnight, then cooled to room temperature. After removal of the solvent under reduced pressure, the residue was separated between EtOAc and water. The organic phase was washed with brine, dried over anhydrous Na₂SO₄, concentrated under reduced pressure and purified with column chromatography (EtOAc: petrol ether=1:5) to give the desired product (1.58 g, 77%).

¹H NMR (CDCl₃): δ 8.24 (1H, d, J=8.8 Hz), 8.06 (1H, s), 7.95 (1H, d, J=6.4 Hz), 2.78 (3H, s).

### Step 3: 5-bromo-6-fluoro-1H-indazole

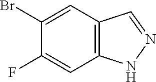 [SMILES: Fc1cc2[nH]ncc2cc1Br]

The solution of 1-(5-bromo-6-fluoro-1H-indazol-1-yl)ethanone (1.58 g, 6.15 mmol) in 3 M a.q. HCl (20 mL) and MeOH (4 mL) was heated at 90° C. for 3 hours, then cooled to room temperature and basified with 1 M a.q. NaOH to pH=10. A colorless solid precipitated out which was filtered and dried in vacuo to provide the desired product (1.22 g, 93%).

¹H NMR (CDCl₃): δ 9.95-10.20 (1H, br), 8.02 (1H, s), 7.97 (1H, d, J=6.4 Hz), 7.28 (1H, s).

### Step 4: 5-bromo-1-(1-(5-ethylpyrimidin-2-yl)piperidin-4-yl)-6-fluoro-1H-indazole

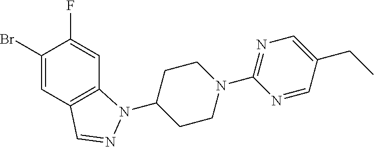 [SMILES: CCc1cnc(N2CCC(n3ncc4cc(Br)c(F)cc43)CC2)nc1]

5-bromo-1-(1-(5-ethylpyrimidin-2-yl)piperidin-4-yl)-6-fluoro-1H-indazole was synthesized from 5-bromo-6-fluoro-1H-indazole (Example 17, step 3) in a similar manner as described in Example 16, step 2.

¹H NMR (CDCl₃): δ 8.19 (2H, s), 7.90-7.92 (2H, m), 7.21-7.24 (1H, m), 4.91-4.95 (2H, m), 4.53-4.58 (1H, m), 3.06-3.13 (2H, m), 2.45 (2H, q, J=7.6 Hz), 2.19-2.30 (2H, m), 2.05-2.09 (2H, m), 1.20 (3H, t, J=7.6 Hz).

### Step 5: 5-(4-(1H-tetrazol-1-yl)phenyl)-1-(1-(5-ethylpyrimidin-2-yl)piperidin-4-yl)-6-fluoro-1H-indazole

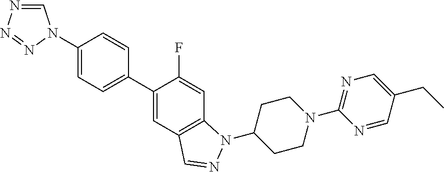 [SMILES: CCc1cnc(N2CCC(n3ncc4cc(-c5ccc(-n6cnnn6)cc5)c(F)cc43)CC2)nc1]

5-(4-(1H-tetrazol-1-yl)phenyl)-1-(1-(5-ethylpyrimidin-2-yl)piperidin-4-yl)-6-fluoro-1H-indazole was synthesized from 5-bromo-1-(1-(5-ethylpyrimidin-2-yl)piperidin-4-yl)-6-fluoro-1H-indazole (Example 17, step 4) and 1-(4-(4,4,5,5-tetramethyl-1,3,2-dioxaborolan-2-yl)phenyl)-1H-tetrazole (Example 2, step 2) in a similar manner as described in Example 2, step 7.

¹H NMR (CDCl₃): δ 9.04 (1H, s), 8.22 (2H, s), 8.05 (1H, s), 7.77-7.83 (5H, m), 7.28-7.31 (1H, m), 4.95-4.99 (2H, m), 4.60-4.68 (1H, m), 3.11-3.18 (2H, m), 2.49 (2H, q, J=7.6 Hz), 2.26-2.37 (2H, m), 2.11-2.16 (2H, m), 1.22 (3H, t, J=7.6 Hz).

### Example 18

### 1-(1-(5-ethylpyrimidin-2-yl)piperidin-4-yl)-6-fluoro-5-(4-(methylsulfonyl)phenyl)-1H-indazole

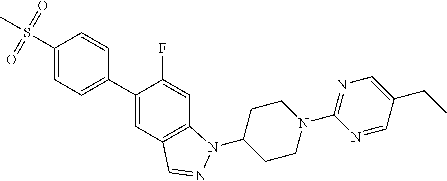 [SMILES: CCc1cnc(N2CCC(n3ncc4cc(-c5ccc(S(C)(=O)=O)cc5)c(F)cc43)CC2)nc1]

1-(1-(5-ethylpyrimidin-2-yl)piperidin-4-yl)-6-fluoro-5-(4-(methylsulfonyl)phenyl)-1H-indazole was synthesized from 5-bromo-1-(1-(5-ethylpyrimidin-2-yl)piperidin-4-yl)-6-fluoro-1H-indazole (Example 17, step 4) and 4,4,5,5-tetramethyl-2-(4-(methylsulfonyl)phenyl)-1,3,2-dioxaborolane in a similar manner as described in Example 1, step 4.

¹H NMR (CDCl₃): δ 8.22 (1H, s), 8.03 (2H, d, J=8.4 Hz), 7.75-7.80 (3H, m), 7.30-7.32 (1H, m), 6.98 (1H, s), 4.95-5.00 (2H, m), 4.60-4.68 (1H, m), 3.14-3.16 (2H, m), 3.13 (3H, s), 2.49 (2H, q, J=7.6 Hz), 2.26-2.37 (2H, m), 2.11-2.16 (2H, m), 1.22 (3H, t, J=7.6 Hz).

### Example 19

### 5-(4-(6-fluoro-5-(4-(methylsulfonyl)phenyl)-1H-indol-1-yl)piperidin-1-yl)-3-isopropyl-1,2, 4-oxadiazole

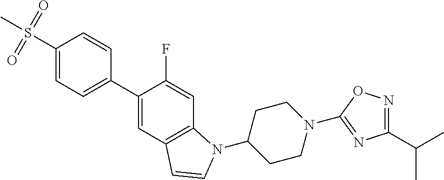 [SMILES: CC(C)c1noc(N2CCC(n3ccc4cc(-c5ccc(S(C)(=O)=O)cc5)c(F)cc43)CC2)n1]

### Step 1: 4-hydroxypiperidine-1-carbonitrile

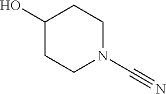

A slurry of sodium bicarbonate (10.5 g, 99.0 mmol) in water (7 mL) was cooled in an ice-bath and a solution of 4-hydroxypiperidine (5.0 g, 49.4 mmol) in dichloromethane (8 mL) was added. With rapid stirring, a solution of cyanogen bromide (6.28 g, 59.3 mmol) in dichloromethane (8 mL) was dropwised over 15 min at 0° C. The ice bath was removed, and the reaction mixture was stirred overnight at room temperature. Sodium carbonate (10 g) was added in order to ensure the completion of neutralization. MgSO₄ (20 g) was added, and the mixture was stirred vigorously for 15 minutes. The resulting suspension was filtered, rinsing with CH₂Cl₂ (200 mL). The solvent removed in vacuo to give the desired product (5.8 g, 93%).

¹H NMR (CDCl₃): δ 3.83-3.93 (1H, m), 3.43-3.50 (2H, m), 3.06-3.13 (2H, m), 1.87-1.97 (2H, m), 1.62-1.70 (2H, m).

### Step 2: 1-(3-isopropyl-1,2,4-oxadiazol-5-yl)piperidin-4-ol

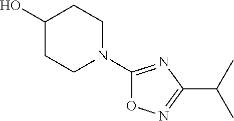

To a magnetically stirred solution of N-hydroxy-isobutyramidine (2.43 g, 24.0 mmol) and 4-hydroxypiperidine-1-carbonitrile (2.50 g, 19.8 mmol) in ethyl acetate (120 mL), ZnCl₂ (1 N in ether, 24 mL, 24.0 mmol) was added in a dropwise fashion over 15 minutes. After stirring for 60 min, the supernatant was decanted and filtered, and the residue was rinsed twice with ether, furnishing a hard white precipitate which was collected by filtration. This material was taken up in con. HCl (12.5 mL), diluted to 4 N with EtOH (25 mL), and refluxed for 1 hour. Upon cooling, a white precipitate was removed by filtration, and then the filtrate was reduced to 10 mL and diluted with 20 mL water. Solid Na₂CO₃ was added until the mixture was basic, CH₂Cl₂ was added, and the resulting mixture was filtered, rinsing with CH₂Cl₂. The organic extracts was separated, dried over MgSO₄, and the solvent was removed in vacuo to afford the desired product (0.5 g, 12%).

¹H NMR (CDCl₃): δ 3.90-3.97 (3H, m), 3.34-3.41 (2H, m), 2.85-2.91 (1H, m), 1.92-1.99 (2H, m), 1.74 (1H, s), 1.60-1.68 (2H, m), 1.27-1.30 (6H, m).

### Step 3: 1-(3-isopropyl-1,2,4-oxadiazol-5-yl)piperidin-4-yl methanesulfonate

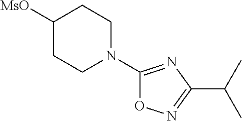 [SMILES: CC(C)c1noc(N2CCC(OS(C)(=O)=O)CC2)n1]

To the mixture of 1-(3-isopropyl-1,2,4-oxadiazol-5-yl)piperidin-4-ol (0.70 g, 3.31 mmol) and triethylamine (0.51 ml, 3.98 mmol) in CH₂Cl₂ (15 mL), methanesulfonyl chloride (0.38 g, 3.31 mmol) was added at 0° C. and stirred overnight at room temperature. Sat. NaHCO₃ solution was added, extracted with CH₂Cl₂. The combined organic layers were washed with water and brine, dried over Na₂SO₄, concentrated in vacuo to give the desired product (0.85 g, 89%).

¹H NMR (CDCl₃): δ 4.96-5.03 (1H, m) 3.78-3.85 (2H, m), 3.06 (3H, s), 2.85-2.92 (1H, m), 2.04-2.11 (2H, m), 1.92-2.02 (2H, m), 1.27-1.29 (6H, m).

### Step 4: 5-(4-(5-bromo-6-fluoro-1H-indol-1-yl)piperidin-1-yl)-3-isopropyl-1,2,4-oxadiazole

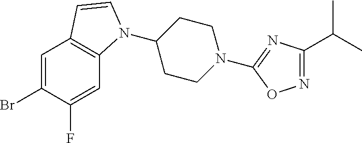 [SMILES: CC(C)c1noc(N2CCC(n3ccc4cc(Br)c(F)cc43)CC2)n1]

5-(4-(5-bromo-6-fluoro-1H-indol-1-yl)piperidin-1-yl)-3-isopropyl-1,2,4-oxadiazole was synthesized from 5-bromo-6-fluoro-1H-indole (Example 6, step 3) and 1-(3-isopropyl-1,2,4-oxadiazol-5-yl)piperidin-4-yl methanesulfonate (Example 19, step 3) in a similar manner as described in Example 1, step 3.

¹H NMR (CDCl₃): δ 7.77 (1H, d, J=6.8 Hz), 7.13-7.17 (2H, m), 6.47 (1H, d, J=3.2 Hz), 4.34-4.40 (2H, m), 4.30 (1H, s), 4.24-4.32 (2H, m), 2.88-2.95 (2H, m), 2.17-2.20 (2H, m), 1.99-2.10 (2H, m), 1.29-1.32 (6H, m).

### Step 5: 5-(4-(6-fluoro-5-(4-(methylsulfonyl)phenyl)-1H-indol-1-yl)piperidin-1-yl)-3-isopropyl-1,2,4-oxadiazole

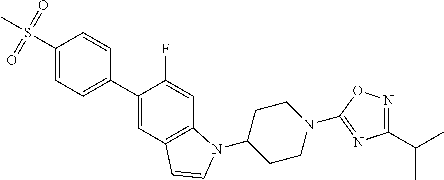 [SMILES: CC(C)c1noc(N2CCC(n3ccc4cc(-c5ccc(S(C)(=O)=O)cc5)c(F)cc43)CC2)n1]

5-(4-(6-fluoro-5-(4-(methylsulfonyl)phenyl)-1H-indol-1-yl)piperidin-1-yl)-3-isopropyl-1,2, 4-oxadiazole was synthesized from 5-(4-(5-bromo-6-fluoro-1H-indol-1-yl)piperidin-1-yl)-3-isopropyl-1,2,4-oxadiazole (Example 19, step 4) and 4,4,5,5-tetramethyl-2-(4-(methylsulfonyl)phenyl)-1,3,2-dioxaborolane in a similar manner as described in Example 1, step 4.

¹H NMR (CDCl₃) δ 8.00-8.02 (2H, m), 7.77-7.80 (2H, m), 7.68 (1H, d, J=7.6 Hz), 7.21 (1H, d, J=3.2 Hz), 7.18 (1H, s), 6.59 (1H, d, J=3.2 Hz), 4.39-4.43 (3H, m), 3.27-3.34 (2H, m), 3.11 (3H, s), 2.91-2.97 (1H, m), 2.21-2.24 (2H, m), 2.08-2.12 (2H, m), 1.30-1.32 (6H, m).

Testing of Compounds of the Invention In Vitro (cAMP Assay)

The functional agonist activities of compounds of the invention were characterized using a cAMP assay with human GPR119 stable transfected Chinese hamster ovary (CHO) cells (American Type Culture Collection, Manassas Va., USA). CHO cells were stable transfected with GPR119/pcDNA3.0 (SC307189, Origene). Transfected cells were then selected and maintained in culture media containing 1200 mg/ml geneticin. Stable clones were obtained by limiting dilution and the expression of human-GPR119 in CHO cells was confirmed by HTRF (Homogeneous Time-Resolved Fluorescence) cAMP assay. The clones generating the best agonist stimulated signal window were selected for the cAMP assay development.

The cells were cultured in Dulbecco's Modified Eagle Medium (Invitrogen Corporation, Carlsbad, Calif., USA) containing 10% Fetal bovine serum, 1% Pen/Strep and 1200 mg/ml G418 and grown in 75 cm2 tissue culture flasks until they reached 75-80% confluence.

The cells were harvested 16 hours prior to assay with 1 ml 0.05% Trypsine, washed with PBS and then plated into 96-well plates (8000 cells/well) containing DMEM medium and 10% BSA. Prior to assay, the cells were washed with assay stimulation buffer (HBSS containing 10 mM IBMX, 20 mM HEPES, 0.1% BSA) twice. Then cells were incubated for 30 min at 37° C. in the absence or presence of varying concentrations of agonists (i.e., the compounds of the present invention) in assay stimulating buffer with 0.1% DMSO. The intracellular levels of cAMP generated in the GPR119 transfected CHO cells were measured using the HTRF kit (CisBio, FR.). In brief, 20 ul d2-labeled cAMP and 20 ul cryptated labeled anti-cAMP antibody were added into 40 ul cells treated with agonist and the plates were incubated for 1 hour at room temperature in the dark. Cells were then transferred into white 96-well plate and the signal generated was measured using Envision (Perkin-Elmer, Norwalk, Conn.). EC50 values were calculated using the GraphPad Prism 5 program.

Biologic Activity

| Compound | Compound structure | EC₅₀ (nm) |
|---|---|---|
| Example 1 | 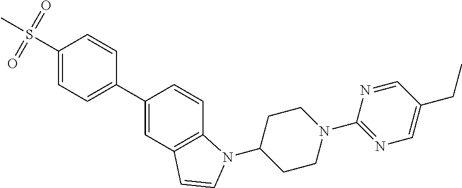 [SMILES: CCc1cnc(N2CCC(n3ccc4cc(-c5ccc(S(C)(=O)=O)cc5)ccc43)CC2)nc1] | <300 |
| Example 2 | 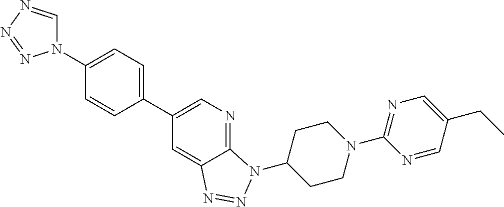 [SMILES: CCc1cnc(N2CCC(n3nnc4cc(-c5ccc(-n6cnnn6)cc5)cnc43)CC2)nc1] | <500 |
| Example 3 | 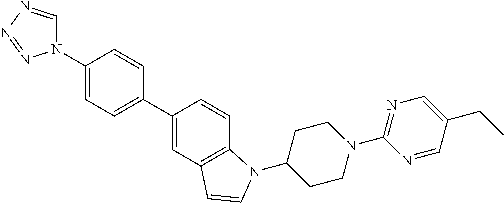 [SMILES: CCc1cnc(N2CCC(n3ccc4cc(-c5ccc(-n6cnnn6)cc5)ccc43)CC2)nc1] | <300 |
| Example 4 | 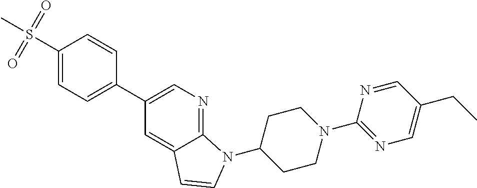 [SMILES: CCc1cnc(N2CCC(n3ccc4cc(-c5ccc(S(C)(=O)=O)cc5)cnc43)CC2)nc1] | NA |
| Example 5 | 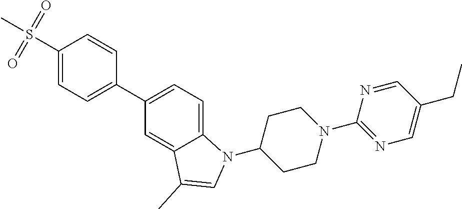 [SMILES: CCc1cnc(N2CCC(n3cc(C)c4cc(-c5ccc(S(C)(=O)=O)cc5)ccc43)CC2)nc1] | <1000 |
| Example 6 | 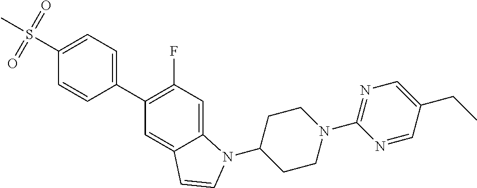 [SMILES: CCc1cnc(N2CCC(n3ccc4cc(-c5ccc(S(C)(=O)=O)cc5)c(F)cc43)CC2)nc1] | <100 |
| Example 7 | 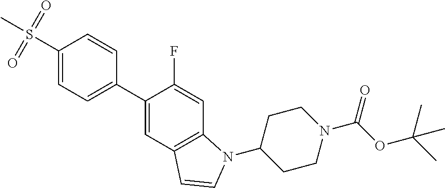 [SMILES: CC(C)(C)OC(=O)N1CCC(n2ccc3cc(-c4ccc(S(C)(=O)=O)cc4)c(F)cc32)CC1] | <100 |
| Example 8 | 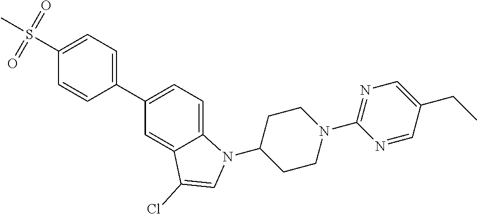 [SMILES: CCc1cnc(N2CCC(n3cc(Cl)c4cc(-c5ccc(S(C)(=O)=O)cc5)ccc43)CC2)nc1] | <500 |
| Example 9 | 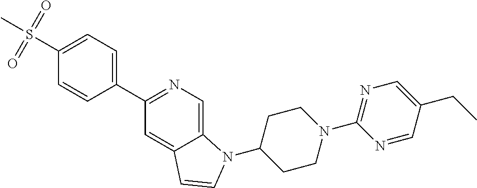 [SMILES: CCc1cnc(N2CCC(n3ccc4cc(-c5ccc(S(C)(=O)=O)cc5)ncc43)CC2)nc1] | <200 |
| Example 10 | 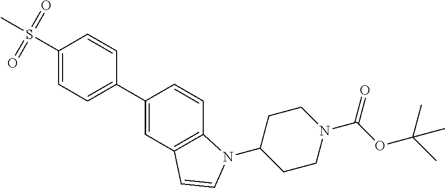 [SMILES: CC(C)(C)OC(=O)N1CCC(n2ccc3cc(-c4ccc(S(C)(=O)=O)cc4)ccc32)CC1] | <200 |
| Example 11 | 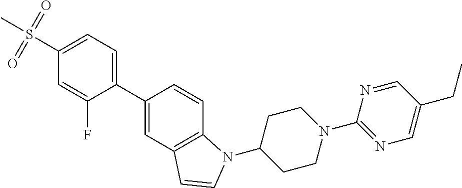 [SMILES: CCc1cnc(N2CCC(n3ccc4cc(-c5ccc(S(C)(=O)=O)cc5F)ccc43)CC2)nc1] | <500 |
| Example 12 | 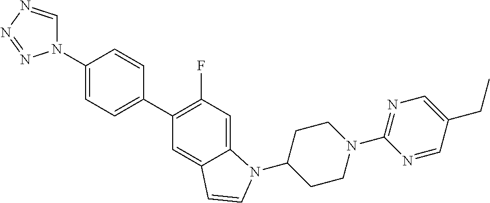 [SMILES: CCc1cnc(N2CCC(n3ccc4cc(-c5ccc(-n6cnnn6)cc5)c(F)cc43)CC2)nc1] | <2000 |
| Example 13 | 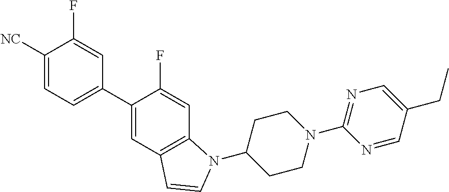 [SMILES: CCc1cnc(N2CCC(n3ccc4cc(-c5ccc(C#N)c(F)c5)c(F)cc43)CC2)nc1] | <500 |
| Example 14 | 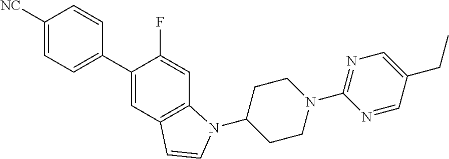 [SMILES: CCc1cnc(N2CCC(n3ccc4cc(-c5ccc(C#N)cc5)c(F)cc43)CC2)nc1] | <500 |
| Example 15 | 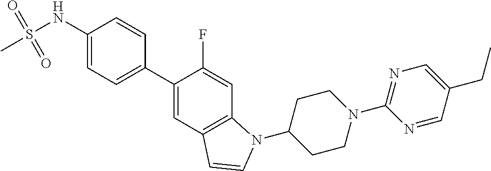 [SMILES: CCc1cnc(N2CCC(n3ccc4cc(-c5ccc(NS(C)(=O)=O)cc5)c(F)cc43)CC2)nc1] | <1000 |
| Example 16 | 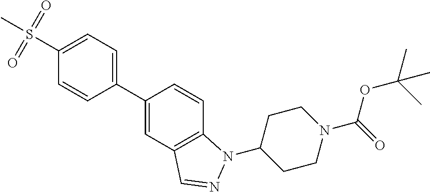 [SMILES: CC(C)(C)OC(=O)N1CCC(n2ncc3cc(-c4ccc(S(C)(=O)=O)cc4)ccc32)CC1] | <300 |
| Example 17 | 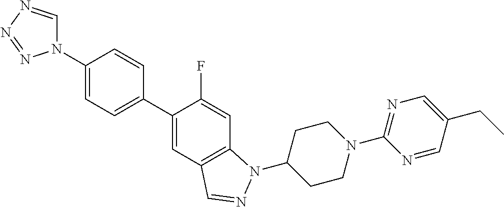 [SMILES: CCc1cnc(N2CCC(n3ncc4cc(-c5ccc(-n6cnnn6)cc5)c(F)cc43)CC2)nc1] | <50 |
| Example 18 | 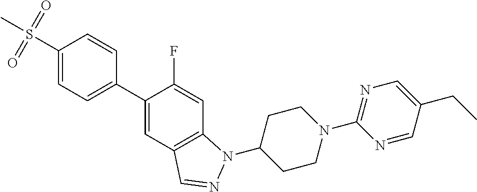 [SMILES: CCc1cnc(N2CCC(n3ncc4cc(-c5ccc(S(C)(=O)=O)cc5)c(F)cc43)CC2)nc1] | <200 |
| Example 19 | 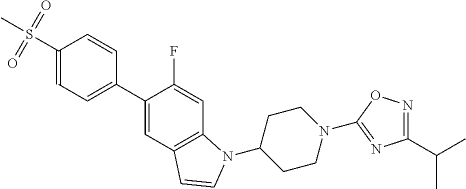 [SMILES: CC(C)c1noc(N2CCC(n3ccc4cc(-c5ccc(S(C)(=O)=O)cc5)c(F)cc43)CC2)n1] | <100 |

1. A compound represented by Formula I: 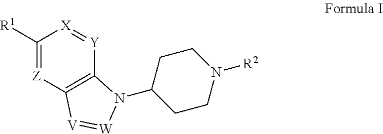
wherein:
R¹ is aryl, unsubstituted or substituted with one or more substituents selected from the group consisting of halogen, alkyl, alkoxy, OCF₃, alkoxycarbonyl, cyano, NHC(O)-alkyl, SO₂-alkyl, SO₂-cycloalkyl, SO₂NH₂, SO₂NH-alkyl, C(O)-alkyl, NO₂, NHS(O)₂-alkyl, SO₂N-(alkyl)₂, —N(alkyl)-SO₂-alkyl, CONH-alkyl, CON-(alkyl)₂, S(O)-alkyl, S(O)-cycloalkyl, C(O)NH₂, triazole, tetrazole, acetyl-piperazine, unsubstituted monocyclic heteroaryl and monocyclic heteroaryl substituted with alkyl;
1,1-dioxo-2,3-dihydro-1H-1-benzo[b]thiophenyl;
monocyclic heteroaryl, unsubstituted or substituted with one or more substituents selected from the group consisting of halogen, SO₂-alkyl, SO₂-cycloalkyl, lower alkyl, triazole, tetrazole, monocyclic heteroaryl with one or two heteroatoms selected from the group consisting of N, O and S; oxo, alkoxy, cyano and hydroxyl; indole, unsubstituted or substituted with one or more substituents selected from the group consisting of lower alkyl, oxo, triazole, tetrazole, SO₂-alkyl and SO₂-cycloalkyl;
benzo[1,3]dioxole, unsubstituted or substituted with one or more substituents selected from the group consisting of alkyl, triazole, tetrazole, oxo, SO₂-alkyl, and SO₂-cycloalkyl;
quinoline, unsubstituted or substituted with one or more substituents selected from the group consisting of lower alkyl, oxo, triazole, tetrazole, SO₂-alkyl and SO₂-cycloalkyl;
pyrrolo[2,3-b]pyridine, unsubstituted or substituted with one or more substituents selected from the group consisting of lower alkyl, oxo, triazole, tetrazole, SO₂-alkyl and SO₂-cycloalkyl;
benzothiophene, unsubstituted or substituted with one or more substituents selected from the group consisting of lower alkyl, oxo, SO₂-alkyl and SO₂-cycloalkyl; or
dioxobenzothiophene, unsubstituted or substituted with one or more substituents selected from the group consisting of lower alkyl, oxo, triazole, tetrazole, SO₂-alkyl and SO₂-cycloalkyl;
R² is benzyl, unsubstituted or substituted with one or more substituents selected from the group consisting of cyano, alkoxy, halogen, hydroxy, OCF₃ and CF₃;
C(O)—O-alkyl, said alkyl is unsubstituted or substituted with one or more substituents selected from the group consisting of halogen, lower alkyl or alkoxy;
C(O)—O—(CH₂)-cycloalkyl, said cycloalkyl is unsubstituted or substituted with one or more substituents selected from the group consisting of halogen, lower alkyl or alkoxy;
C(O)—O—(CH₂)-phenyl, said phenyl is unsubstituted or substituted with one or more substituents selected from the group consisting of halogen, CF₃, cyano or NO₂;
heteroaryl, unsubstituted or substituted with one or more substituents selected from the group consisting of halogen, lower alkyl, cycloalkyl or alkoxy;
(CH₂)-heteroaryl, said heteroaryl is unsubstituted or substituted with one or more substituents selected from the group consisting of halogen, lower alkyl, cycloalkyl or alkoxy;
C(O)-lower alkyl, said alkyl is unsubstituted or substituted with one or more substituents selected from the group consisting of halogen, lower alkyl or alkoxy;
C(O)(CH₂)-cycloalkyl, said cycloalkyl is unsubstituted or substituted with one or more substituents selected from the group consisting of halogen, lower alkyl or alkoxy;
C(O)(CH₂)-phenyl, said phenyl is unsubstituted or substituted with one or more substituents selected from the group consisting of halogen or alkoxy;
C(O)-heteroaryl, said heteroaryl is unsubstituted or substituted with one or more substituents selected from the group consisting of halogen, lower alkyl or alkoxy;
C(O)-aryl, said aryl is unsubstituted or substituted with one or more substituents selected from the group consisting of halogen, lower alkyl or alkoxy;
CH₂-difluorobenzodioxole; or
SO₂-lower alkyl, said alkyl is unsubstituted or substituted with one or more substituents selected from the group consisting of halogen, lower alkyl or alkoxy; and
n is 0, 1 or 2;
in the moiety of 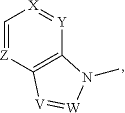
X, Y, Z, V and W are independently selected from N, or CR³; and the moiety is optionally substituted with one or more substituents selected from halogen, cyano, optionally substituted alkyl, cycloalkyl and alkoxy;
R³ is hydrogen, halogen, alkyl, hydroxy or alkoxy;
or a pharmaceutically acceptable salt, solvate, poly-morph, tautomer or prodrug thereof.

2. A compound according to claim 1, wherein in R¹, the aryl is monocyclic aryl; and in the substitutents and in R³, each alkyl is C₁₋₆ alkyl, each cycloalkyl is C₃₋₅ cycloalkyl, and each alkoxy is C₁₋₆ alkoxy; or a pharmaceutically acceptable salt, solvate, poly-morph, tautomer or prodrug thereof.

3. A compound according to claim 1, wherein in R², the heteroaryl is monocyclic heteroaryl with at least one heteroatoms of N, S and O;
and in the substitutents, each alkyl is C₁₋₆ alkyl, each cycloalkyl is C₃₋₅ cycloalkyl, and each alkoxy is C₁₋₆ alkoxy; or a pharmaceutically acceptable salt, solvate, poly-morph, tautomer or prodrug thereof.

4. A compound according to claim 1 represented by the following formula (II): 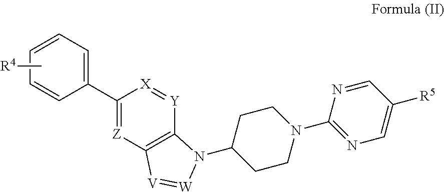
wherein:
R⁴ is at least one group selected from the group consisting of —SO₂C₁₋₄alkyl, —SO₂C₃₋₅cycloalkyl, —NHS(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen;
R⁵ is C₁₋₄alkyl, C₁₋₄alkoxy, halogen, C₃₋₆cycloalkyl or heterocyclic;
or a pharmaceutically acceptable salt, solvate, poly-morph, tautomer or prodrug thereof.

5. A compound according to claim 4, wherein R⁴ is one group at the ortho-position, meta-position or para-position to the other substituent of phenyl; or a pharmaceutically acceptable salt, solvate, poly-morph, tautomer or prodrug thereof.

6. A compound according to claim 2 represented by the following formula (III): 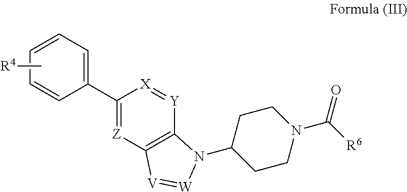
wherein:
R⁴ is at least one group selected from the group consisting of —SO₂alkyl, —SO₂cycloalkyl, —NHS(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen; and
R⁶ is C₁₋₆ alkyl, monocyclic aryl, monoheteroaryl, C₁₋₆ alkoxy, C₃₋₆ cycloalkyl or C₁₋₆ heterocyclic with one or more heteroatoms selected from N, O and S;
or a pharmaceutically acceptable salt, solvate, poly-morph, tautomer or prodrug thereof.

7. A compound according to claim 6, wherein R⁴ is one group at the ortho-position, meta-position or para-position to the other substituent of phenyl; or a pharmaceutically acceptable salt, solvate, poly-morph, tautomer or prodrug thereof.

8. A compound according to claim 3 represented by the following formula (IV): 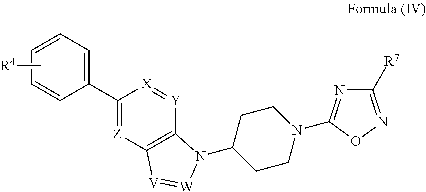
wherein:
R⁴ is at least one group selected from the group consisting of —SO₂alkyl, —SO₂cycloalkyl, —NHS(O)₂-alkyl, —SO₂N-(alkyl)₂, —SO₂NH₂, —SO₂NH-alkyl, —N(alkyl)-SO₂-alkyl, triazole, tetrazole, oxazole, thiazole, oxadiazole, thiodiazole, cyano and halogen;
R⁷ is C₁₋₆alkyl, C₁₋₆alkoxy, halogen, monocyclic aryl, monoheteroaryl, C₃₋₆ cycloalkyl or C₁₋₆heterocyclic with one or more heteroatoms selected from N, O and S;
or a pharmaceutically acceptable salt, solvate, poly-morph, tautomer or prodrug thereof.

9. A compound according to claim 8, wherein R⁴ is one group at the ortho-position, meta-position or para-position to the other substituent of phenyl; or a pharmaceutically acceptable salt, solvate, poly-morph, tautomer or prodrug thereof.

10. A compound according to claim 1, represented by any of the following formulae: 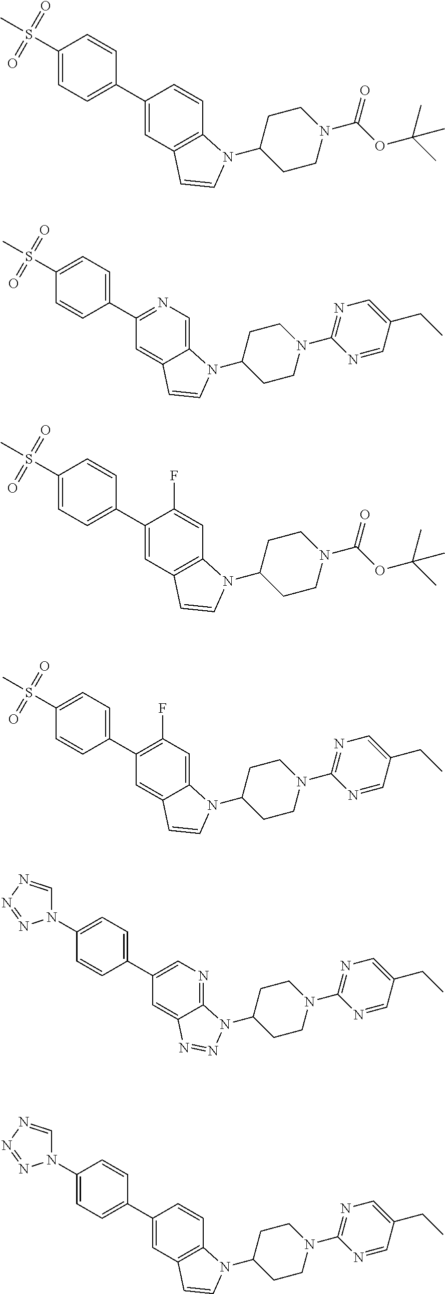 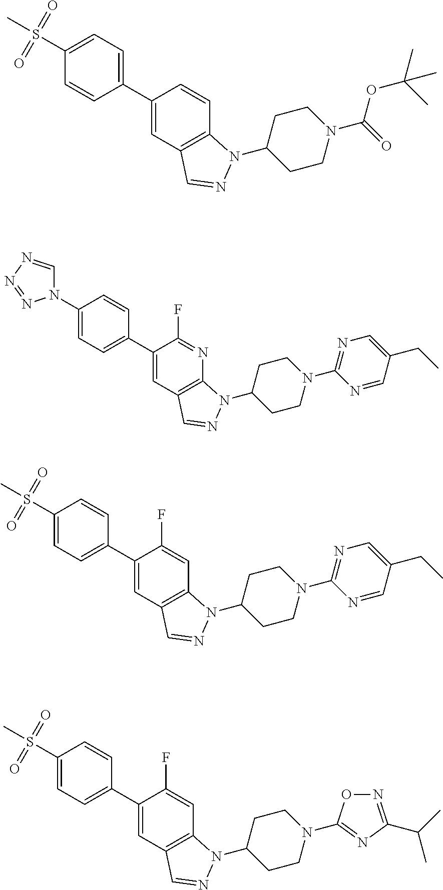
or a pharmaceutically acceptable salt, solvate, poly-morph, tautomer or prodrug thereof.

11.-13. (canceled)

14. A pharmaceutical composition comprising at least one compound according to claim 1 or a pharmaceutically acceptable salt, solvate, poly-morph, tautomer or prodrug thereof and a pharmaceutically acceptable carrier.

15.-17. (canceled)

18. A method for stimulating the release of endogenous insulin from an isolet beta-cell comprising the contact of a compound of claim 1 or a pharmaceutically acceptable salt, solvate, poly-morph, tautomer or prodrug thereof with the cell.

19. A method for the treatment of a metabolic-related disorder in an individual comprising administering to said individual in need of such treatment a therapeutically effective amount of a compound according to any of the claim 1 or a pharmaceutically acceptable salt, solvate, poly-morph, tautomer or prodrug thereof.

20. The method according to claim 19 wherein said individual is a mammal.

21. The method according to claim 20, wherein said mammal is a human.

22. The method according to claim 19 wherein said metabolic-related disorder is selected from the group consisting of Type I diabetes, Type II diabetes, inadquate glucose tolerance, insulin resistance, hyperglycemia, hyperlipidemia, hypertriglyceridemia, hypercholesterolemia, dyslipidemia, obesity and syndrome X.
